(19) United States
(12) Patent Application Publication
(10) Pub. No.: US 2010/0308991 A1
(43) Pub. Date: Dec. 9, 2010

(54) COMMUNICATION SYSTEM FOR HEADS-UP DISPLAY

(75) Inventors: JONATHAN D. ADAMS, Shaker Heights, OH (US); JOSEPH BIRLI, Munson, OH (US); GREG SKILLICORN, Akron, OH (US); GARY L. CLAYPOOLE, West Chester, OH (US); ROBERT L. LIVINGSTON, JR., Cleveland, OH (US)

Correspondence Address: FAY SHARPE LLP, 1228 Euclid Avenue, 5th Floor, The Halle Building, Cleveland, OH, 44115, US

(73) Assignee: Undersea Sensor Systems. Inc.

(21) Appl. No.: 12/856,713

(22) Filed: Aug. 16, 2010

Related U.S. Application Data
(62) Division of application No. 11/705,317, filed on Feb. 12, 2007.

(51) Int. Cl. G08B 1/08; A61M 16/06; F16J 15/02

(52) U.S. Cl. 340/539.12; 128/206.21; 277/316

(57) ABSTRACT

A breathing apparatus designed to display at least one condition associated with the breathing apparatus to the user of the breathing apparatus. The breathing apparatus includes a breathing mask, a display assembly and a sensor assembly. The display assembly includes at least one display designed to display at least one condition of the display assembly and/or the sensor assembly. The breathing apparatus design can be designed to enable a third party to view at least one condition of the display assembly and/or the sensor assembly while a user is wearing the breathing mask. The breathing apparatus can include a sealing arrangement for forming a seal between a circuit board and/or other electronic components and a housing of the display assembly and/or the sensor assembly.

The present invention is a divisional application of U.S. patent application Ser. No. 11/705,317 tiled Feb. 12, 2007. The present invention claims priority on U.S. Provisional Application Ser. No. 60/772,020 tiled Feb. 10, 2006, entitled “Communications System for Heads-Up Display,” all of which is incorporated herein by reference. The present invention also claims priority on U.S. Provisional Application Ser. No. 60/772,452 tiled Feb. 10, 2006, entitled “Heads-Up Display For A Self-Contained Breathing Apparatus,” all of which is also incorporated herein by reference.

The present invention is directed to a mask, and more particularly to a mask that provides air to a wearer of the mask, and even more particularly to a mask that supplies air to a user and which also provides the user and/or a third party information about one or more components of the user's mask, air supply status for the user's mask, battery supply for the user's mask, and/or information about one or more mates to the user's mask.

### BACKGROUND OF THE INVENTION

Self-contained breathing apparatuses (SCBAs) are typically used to provide a safe breathing gas supply to a wearer or user thereof. As such, SCBAs typically include a breathing mask in fluid communication with a breathing gas supply such as, but not limited to, a breathing gas tank. Configured as such, SCBAs are commonly employed by first responders, safety personal and the like (e.g., firefighters, military, police, security personnel, rescue personnel, maintenance and repair personnel, etc.) when operating in unsafe or potentially unsafe environments (e.g., fighting fires, cleaning chemical spills, working within environments that contain hazardous gases, microbes or other airborne contaminants, etc.). As such, it is vital that the amount of breathing gas remaining in the breathing gas supply be known to the user of the mask while the SCBA is in use by the user.

In view of the current state of the art regarding protective masks, there is a need for a mask that supplies breathing air to a user and provides information to a user and/or to a third party about one or more components of the user's mask, air supply status for the user's mask, battery supply for the user's mask, and/or information about one or more mates to the user's mask.

### SUMMARY OF THE INVENTION

The present invention is directed to a breathing apparatus that supplies breathing air to a user and provides information to a user and/or to a third party about one or more components of the user's breathing apparatus, air supply status for the user's breathing apparatus, battery supply for the user's breathing apparatus, and/or information about one or more mates to the user's breathing apparatus.

In one non-limiting aspect of the present invention, there is provided a breathing apparatus designed to display one or more conditions associated with the breathing apparatus to a wearer or user of the breathing apparatus. Such conditions can include, but are not limited to, status of breathing apparatus (e.g., off, on, low power mode, etc.), power level of breathing apparatus, air supply of the breathing apparatus, communication status of the breathing apparatus, time of use of the breathing apparatus, remaining use time of the breathing apparatus, GPS or other location information of breathing apparatus, whether one or more mates for the breathing apparatus are in communication with and/or close proximity to the breathing apparatus, etc. In one non-limiting embodiment of the invention, the breathing apparatus includes 1) a breathing gas supply, 2) a breathing mask associated with and in fluid communication with the breathing gas supply, 3) at least one display assembly on the breathing mask that includes one or more displays associated with at least one condition of the breathing apparatus, 4) at least one sensor assembly that includes at least one sensor associated with at least one condition of the breathing apparatus, and 5) at least one sensor transceiver assembly that is at least partially in communication with the at least one display assembly. As can be appreciated, the breathing apparatus can include less components. As can also be appreciated, the breathing apparatus can include other or additional components. In one non-limiting aspect of this embodiment, the at least one sensor transceiver assembly can be designed to receive at least one data message that is at least partially based on at least one condition sensed by at least one sensor and then transmits the at least one message and/or another data message to at least one display assembly. In another and/or alternative non-limiting aspect of this embodiment, the at least one display assembly can be designed to 1) receive at least one data message, 2) transmit at least one data message and/or another data message to at least one sensor transceiver assembly to acknowledge receipt of the data message, and/or 3) display at least one condition associated with the breathing apparatus on the one or more displays based at least in part on at least one data message and/or another data message. In still another and/or alternative non-limiting aspect of this embodiment, at least one sensor transceiver transmits information obtained from at least one sensor regarding a pressure level of the breathing gas associated with at least one breathing gas supply. The transmitted information regarding pressure level can be transmitted either intermittently or continuously via a wireless or non-wireless arrangement to at least one display assembly. The display assembly can include or be connected to a receiver or transreceiver when the information is transmitted wirelessly; however, this is not required. A control circuit can be used to control the transmission of information that is ultimately received by the display assembly; however, this is not required. The display assembly can include at least one display to enable a user and/or nonuser of the breathing apparatus to be informed of the breathing gas level associated with the breathing gas supply; however, this is not required.

In another and/or alternative non-limiting aspect of the present application, there is provided a breathing apparatus that includes at least one display assembly, and wherein the at least one display assembly includes a housing having at least one battery compartment to enable at least one battery or power supply to be positioned in one or more locations on/in the housing.

In still another and/or alternative non-limiting aspect of the present application, there is provided a breathing apparatus that includes at least one display housing having at least one arrangement designed to at least partially allow one or more displays to be viewed by a user when the breathing apparatus is being worn by the user and to also include at least one arrangement designed to at least partially allow one or more displays to be viewed by a nonuser of the breathing apparatus. In one non-limiting aspect of this embodiment, the breathing apparatus of the present invention can be designed to allow nonusers of the breathing apparatus to view one or more displays of a user's breathing apparatus while the breathing apparatus is worn by the user by viewing the one or more displays of a user's breathing apparatus through a transparent protective shield of the breathing apparatus. As can be appreciated, other or additional arrangements can be designed in the breathing apparatus to enable a nonuser of the breathing apparatus to view one or more displays of a user's breathing apparatus while the breathing apparatus is worn by the user.

In yet another and/or alternative non-limiting aspect of the present application, there is provided a breathing apparatus that includes at least one seal arrangement positioned between one or more components of a display housing of a display assembly in a manner that the sealing arrangement does not cover or impair information displayed on one or more displays. In one non-limiting aspect of this embodiment, the sealing arrangement includes 1) a sealing material that can be applied to a printed circuit board inside and/or about the display housing, and/or 2) a sealing material that can be at least partially positioned between two or more components of the display housing. As can be appreciated, the sealing arrangement can be included in the display housing in other or additional ways. In one non-limiting configuration, at least a portion of the sealing material can be inserted through one or more gaps between a printed circuit board and one or more housing components to at least partially form a seal. In another and/or alternative non-limiting configuration, the sealing material can be formulated to as to at least partially flow into contact with one or more components of the housing to thereby at least partially form a seal with the one or more housing components and/or one or more components in the display housing.

In yet another and/or alternative non-limiting aspect of the present application, there is provided a breathing apparatus that includes an elongated retaining member designed to at least partially secure one or more components of a HUD assembly inside a breathing mask of the breathing apparatus. In one non-limiting embodiment of the invention, the elongated retaining member can be coupled to one or more end portions of the HUD assembly. In another and/or alternative non-limiting embodiment of the invention, the elongated retaining member can be configured to engage a rim around the periphery of a protective shield inside the breathing mask to at least partially secure one or more components of the HUD assembly to the breathing mask.

In still yet another and/or alternative non-limiting aspect of the present application, there is provided a breathing apparatus that includes a controller that causes one or more components of the breathing apparatus to power down or power to a lower energy consumption state so as to consume less energy when it is determined by one or more parameters (e.g., some time of non-use of one or more components of the breathing apparatus, detection of no air consumption, detection that the breathing apparatus is not on a user, no signal transmission and/or reception after some period of time, etc.) have been satisfied.

In another and/or alternative non-limiting aspect of the present application, there is provided a mounting arrangement for the HUD assembly to enable the HUD assembly to be easily and conveniently secured to the inside of the mask. The mounting arrangement can include at least one retaining member that is connected to one or more portions of the HUD assembly. The retaining member can be then secured to the inside surface of a mask thereby at least partially securing the HUD assembly to the mask.

In still another and/or alternative non-limiting aspect of the present application, there is provided a HUD assembly that enables the user of the mask and/or a nonuser of the mark to be notified about one or more conditions of the SCBA. In one non-limiting embodiment of the invention, the HUD assembly includes one or more displays that can be viewed by the user of the mask. The HUD assembly can also include one or more windows and/or other arrangements that can enable a third party to also view one or more of the displays that are viewed by the user. In another and/or alternative embodiment of the invention, the HUD assembly has one or more displays that are positioned in the mask such that both a user and a third party can view one or more of the displays in the mask. In still another and/or alternative embodiment of the invention, the HUD assembly has one or more displays that can only be viewed by the user of the mask, and one or more separate displays that can be only viewed by a third party when the user is wearing the mask. These two separate displays can convey the same or similar information regarding one or more conditions of the SCBA.

In yet another and/or alternative non-limiting aspect of the present application, there is provided a method of sealing one or more components on the housing of a HUD assembly. The method of sealing can be used to form a seal between electronic components and the inside surfaces of a housing of the HUD assembly.

In still yet another and/or alternative non-limiting aspect of the present application, there is provided a method of displaying at least one condition associated with a breathing apparatus to a user of the breathing apparatus. In one non-limiting embodiment of the present invention, the method of displaying at least one condition associated with a breathing apparatus includes 1) obtaining/receiving at least one data message based at least partially on at least one condition associated with the breathing apparatus, 2) transmitting at least one data message to at least one display assembly in a face mask of the breathing apparatus, 3) receiving at least one data message at the display assembly, 4) acknowledging receipt of at least one data message at the display assembly by transmitting at least one other data message to one or more components of the breathing apparatus, and 5) displaying one or more conditions associated with the breathing apparatus based at least partially on the at least one data message received by the display assembly.

In another and/or alternative non-limiting aspect of the present application, there is provided a method of displaying one or more conditions associated with the SCBA.

In still another and/or alternative non-limiting aspect of the present application, there is provided a method of a sensor assembly communicating with a HUD assembly.

In yet another and/or alternative non-limiting aspect of the present application, there is provided a method of establishing a connection between the sensor assembly and the HUD assembly.

In still yet another and/or alternative non-limiting aspect of the present application, there is provided a method of reestablishing a connection between the sensor assembly and the HUD assembly.

In another and/or alternative non-limiting aspect of the present application, there is provided a method of conducting a low power and/or a high power search for a mate to a SCBA.

In still another and/or alternative non-limiting aspect of the present application, there is provided a method of determining whether one or more components of a SCBA should be shutdown based on a power level of a power supply.

In yet another and/or alternative non-limiting aspect of the present application, there is provided a method of determining whether a warning should be displayed based on a power level of a power supply and/or a pressure level in the gas supply.

Unless expressly excluded herein, all combinations and sub-combinations of the aspects and embodiments of the invention set forth above are intended to be within the scope of the present invention.

These and other aspects of a communication system for a heads-up display (HUD) associated with a self-contained breathing apparatus (SCBA) will become apparent to those skilled in the art upon reading and following the description of the invention taken together with the accompanied drawings:

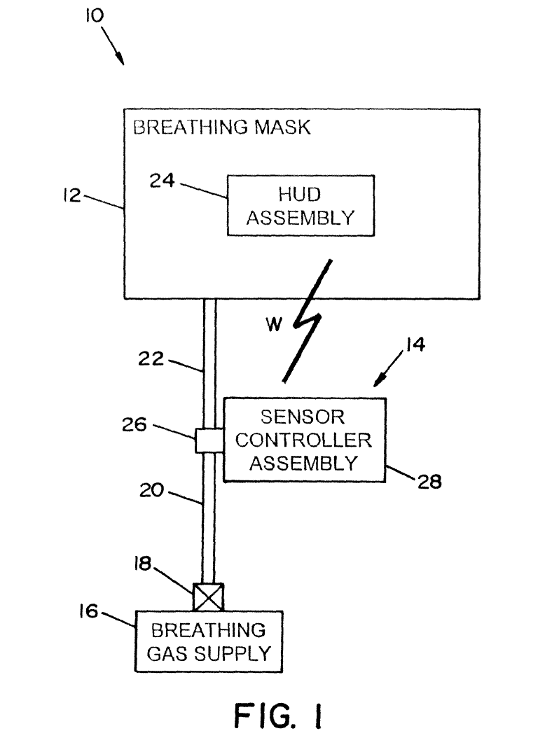

FIG. 1 is a block diagram of one non-limiting embodiment of an SCBA in accordance with the present invention;

FIG. 1A is a drawing illustrating one non-limiting embodiment of an SCBA in accordance with the present invention;

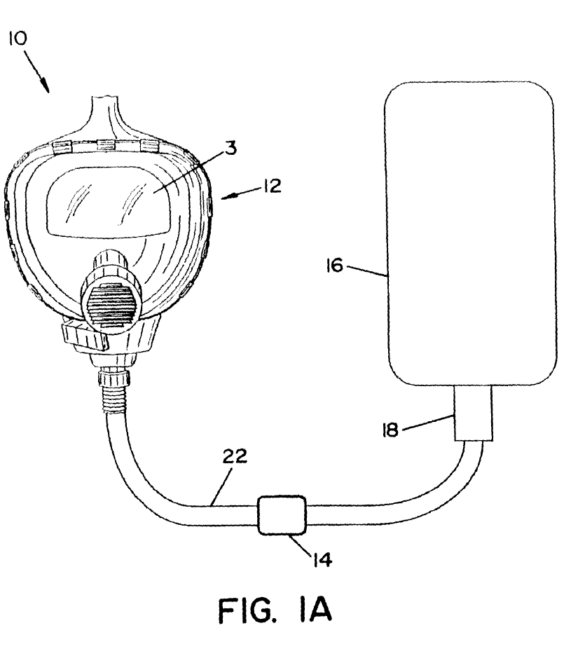

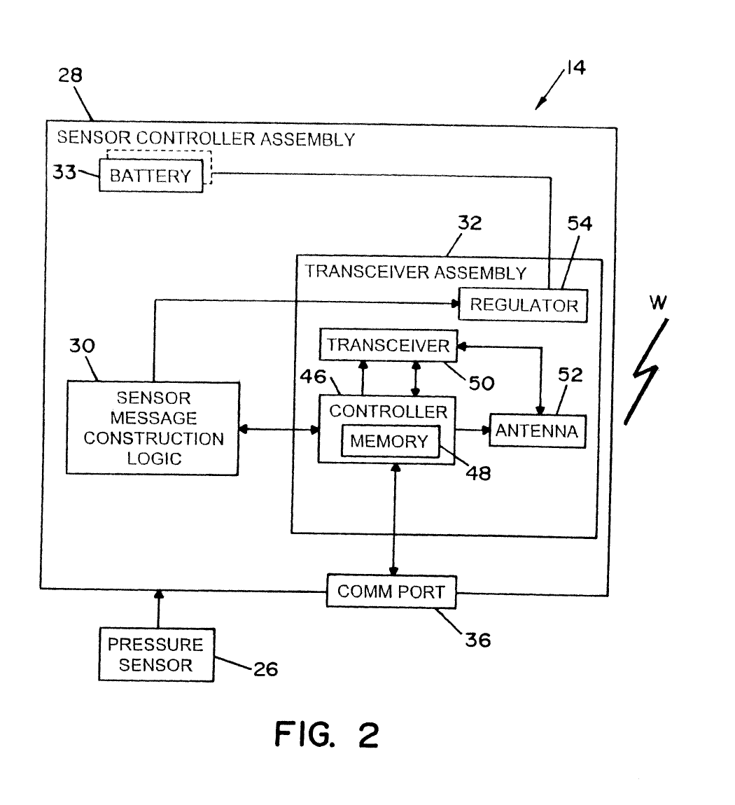

FIG. 2 is a block diagram of one non-limiting embodiment of a sensor controller assembly associated with a sensor assembly of an SCBA;

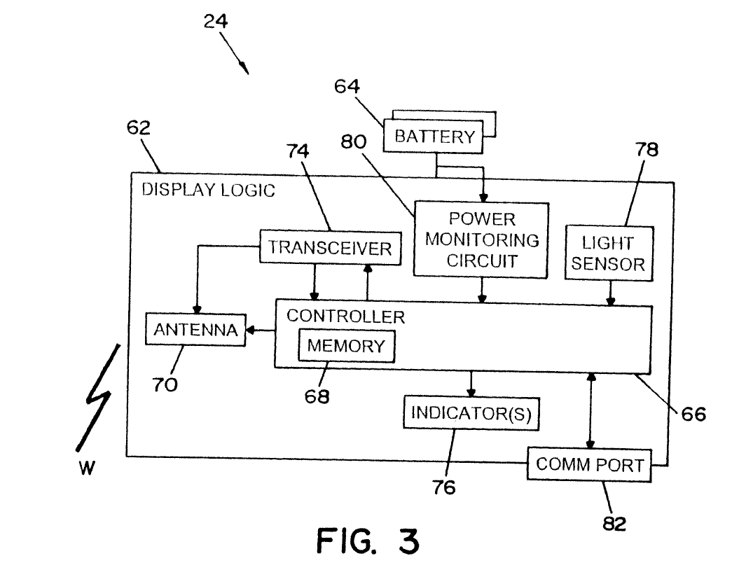

FIG. 3 is a block diagram of one non-limiting embodiment of a HUD assembly associated with a breathing mask of an SCBA;

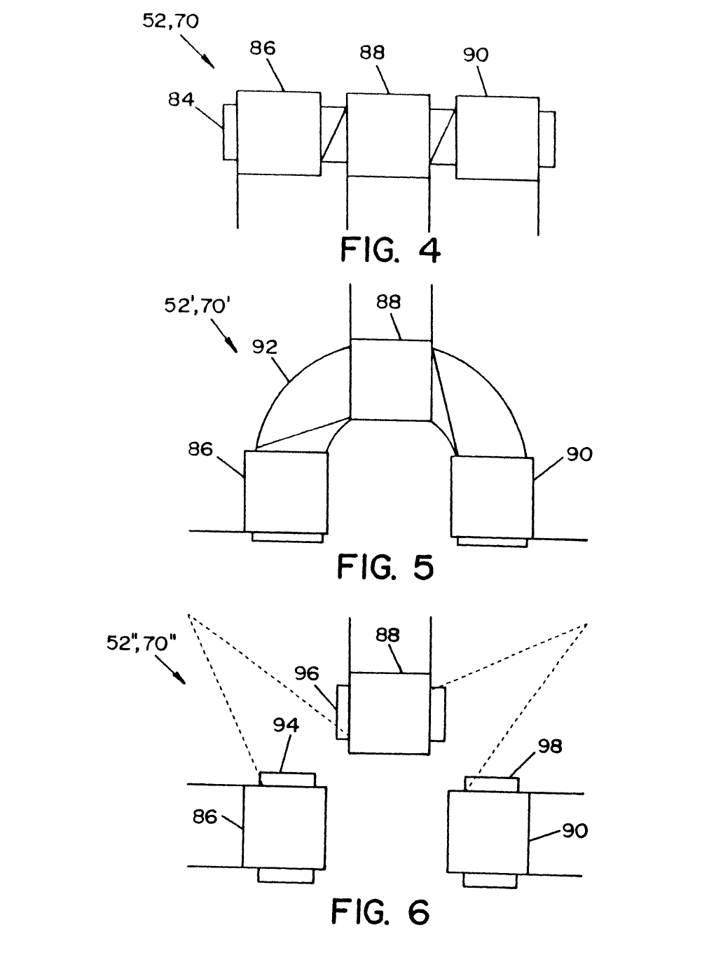

FIG. 4 is a diagram of one non-limiting embodiment of an antenna associated with a sensor controller assembly and/or a HUD assembly;

FIG. 5 is a diagram of one non-limiting embodiment an antenna associated with a sensor controller assembly and/or a HUD assembly;

FIG. 6 is a diagram of one non-limiting embodiment of an antenna associated with a wireless sensor controller assembly and/or a HUD assembly;

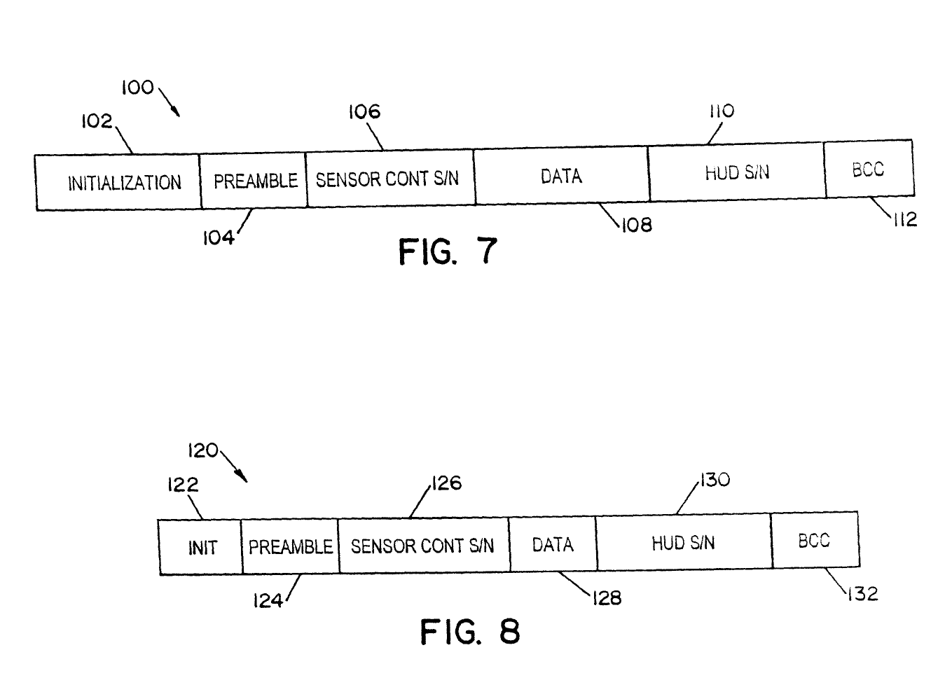

FIG. 7 shows one non-limiting embodiment of a sensor data message sequence that can be transmitted from a sensor controller assembly of an SCBA to a HUD assembly;

FIG. 8 shows one non-limiting embodiment of a display data message sequence that can be transmitted from a HUD assembly of an SCBA to a sensor controller assembly;

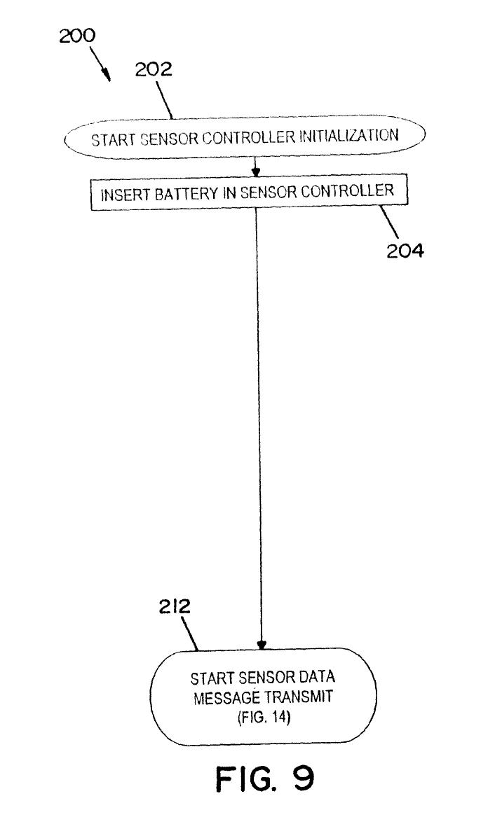

FIG. 9 is a flow chart of one non-limiting embodiment of an initialization process for a sensor controller assembly of an SCBA;

FIG. 10 is a flow chart of one non-limiting embodiment of a sensor data message transmit process in a sensor controller assembly of an SCBA;

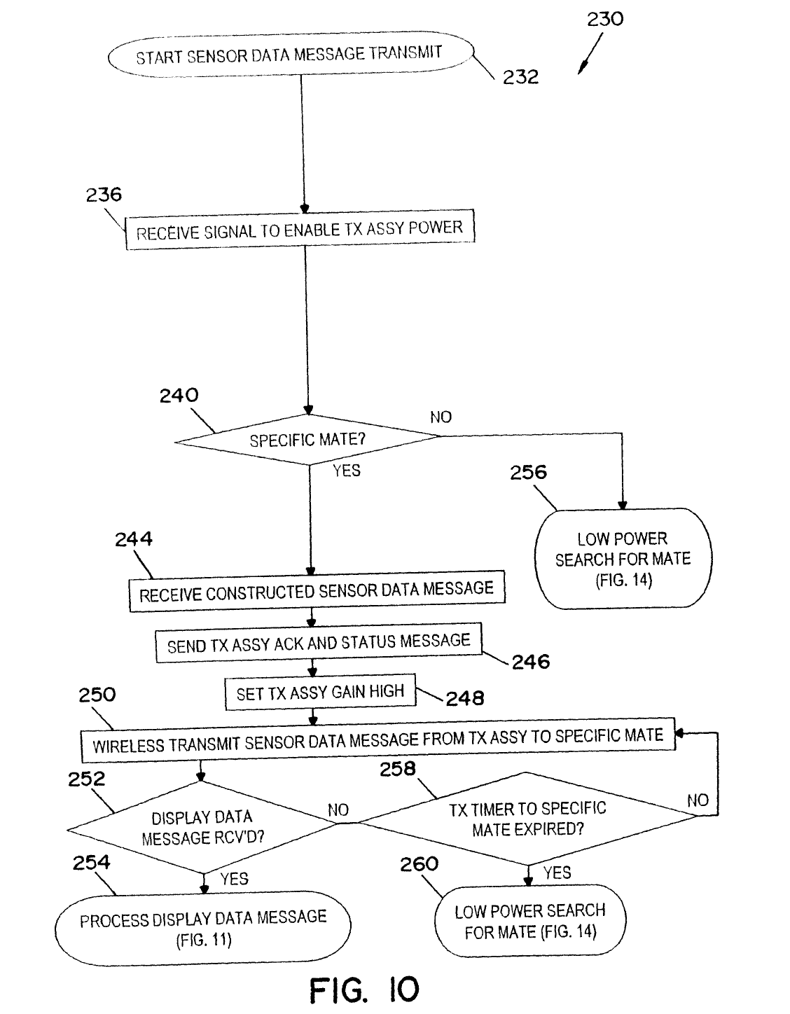

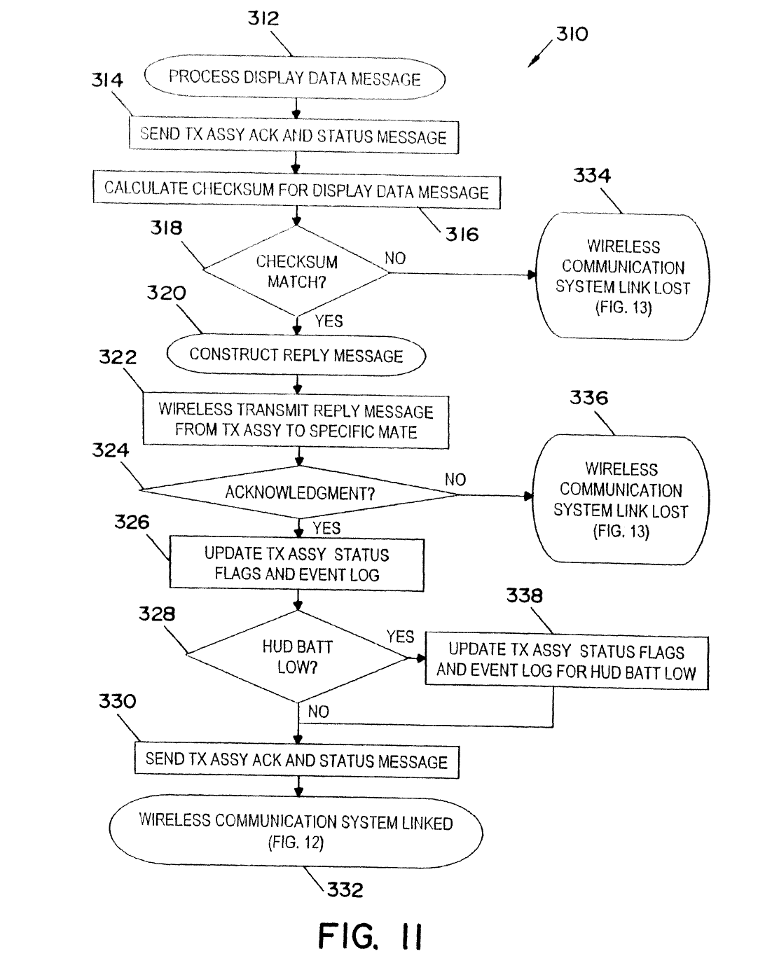

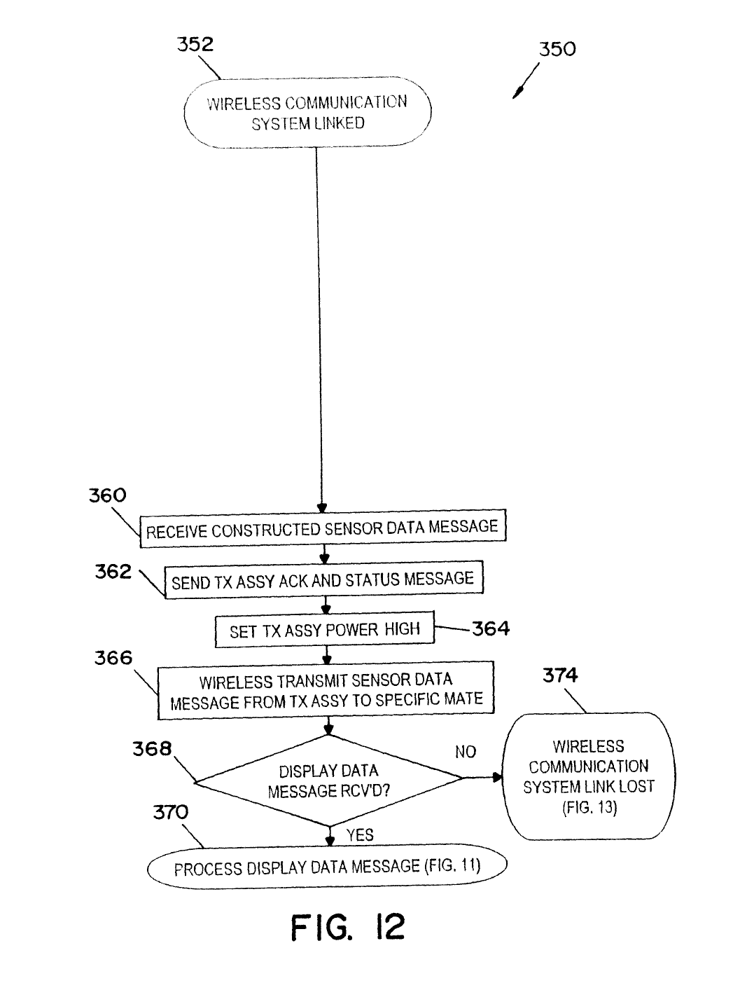

FIG. 11 is a flow chart of one non-limiting embodiment of a method of processing a display data message associated with a sensor controller assembly of an SCBA;

FIG. 12 is a flow chart of one non-limiting embodiment of a communication system linked process associated with a sensor controller assembly of an SCBA;

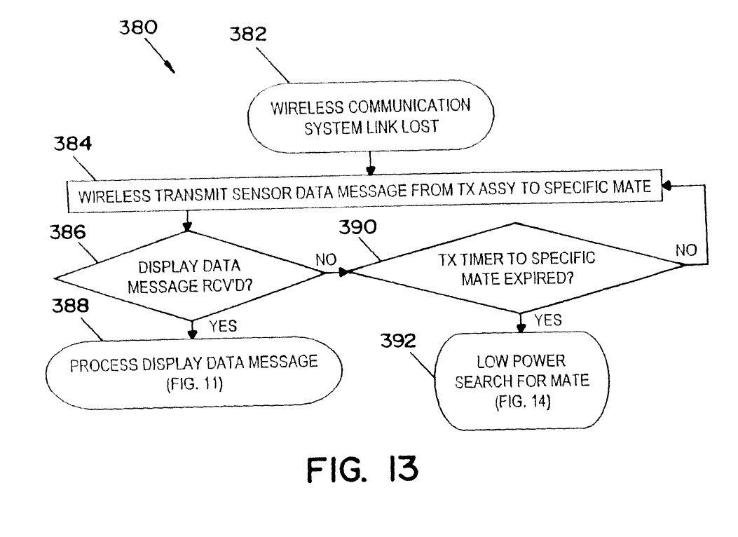

FIG. 13 is a flow chart of one non-limiting embodiment of a communication system link lost process associated with a sensor controller assembly of an SCBA;

FIG. 14 is a flow chart of one non-limiting embodiment of a low power search for mate process associated with a sensor controller assembly of an SCBA;

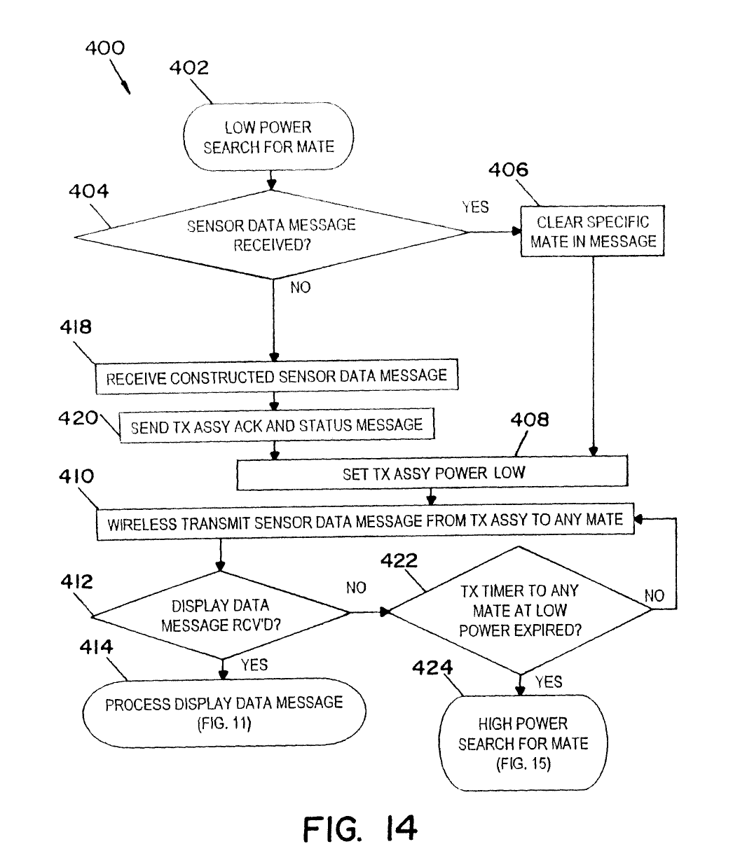

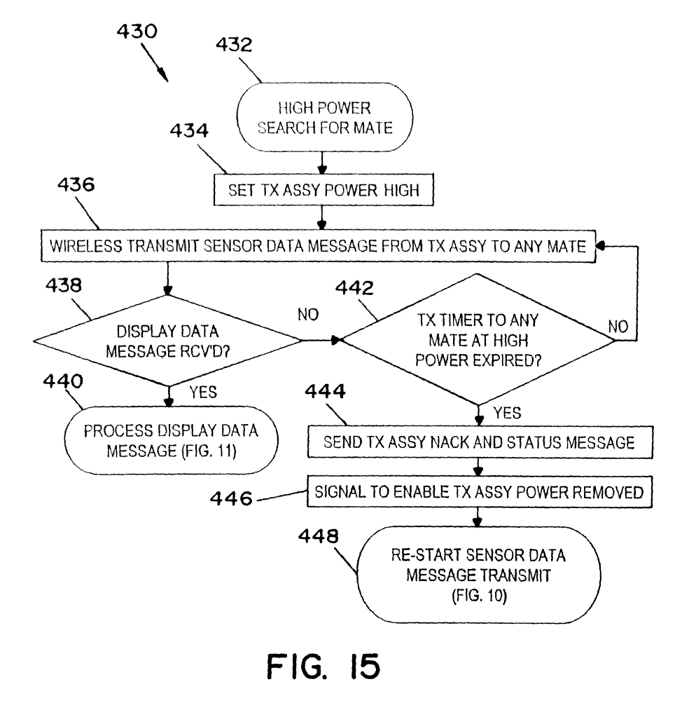

FIG. 15 is a flow chart of one non-limiting embodiment of a high power search for mate process associated with a sensor controller assembly of an SCBA;

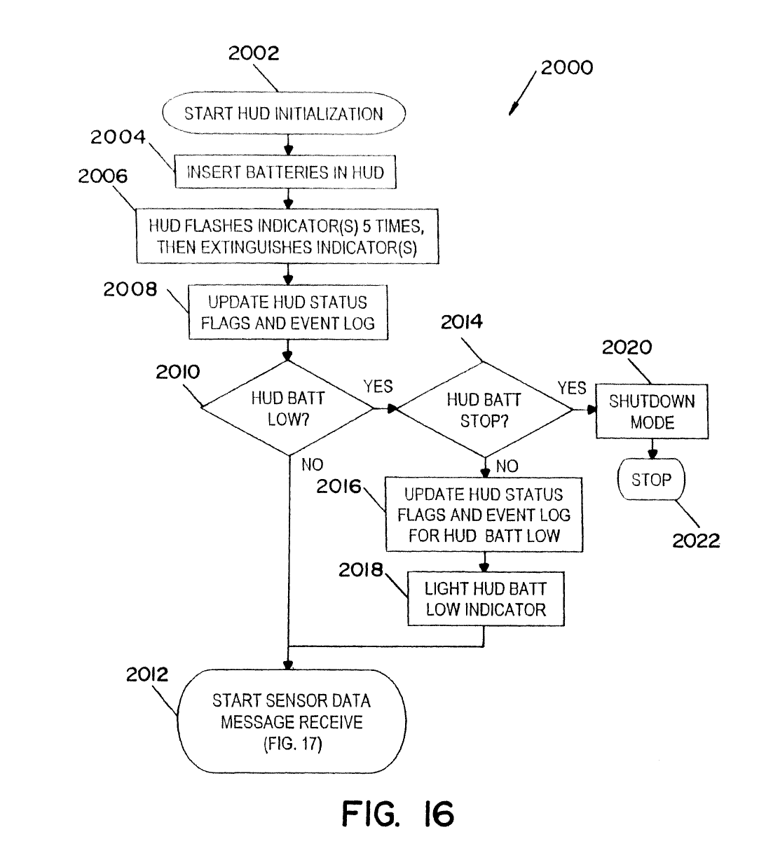

FIG. 16 is a flow chart of one non-limiting embodiment of an initialization process for a HUD assembly of an SCBA;

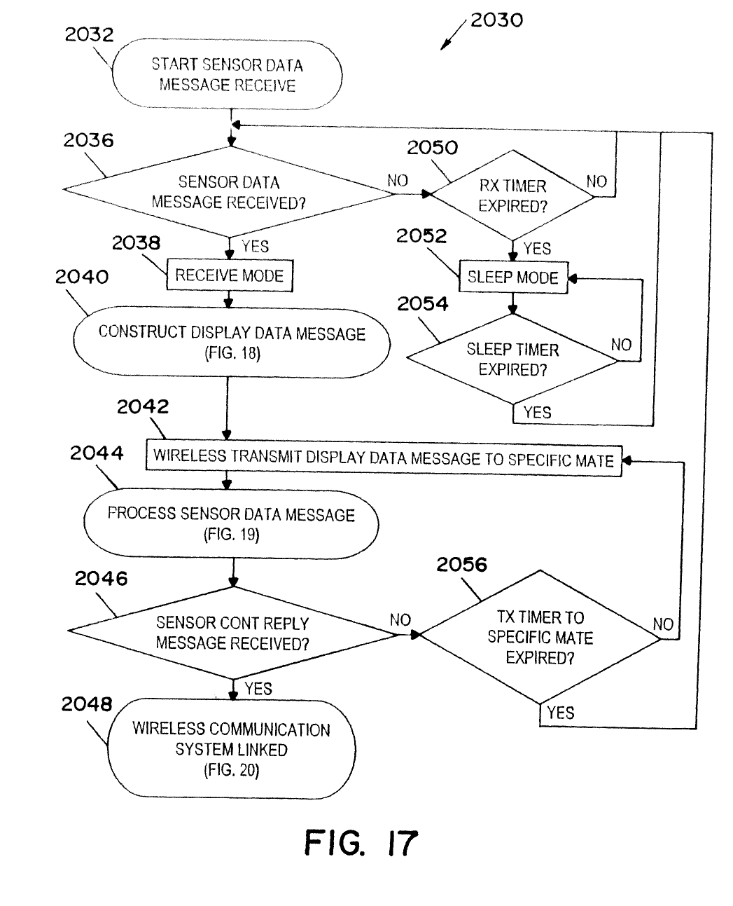

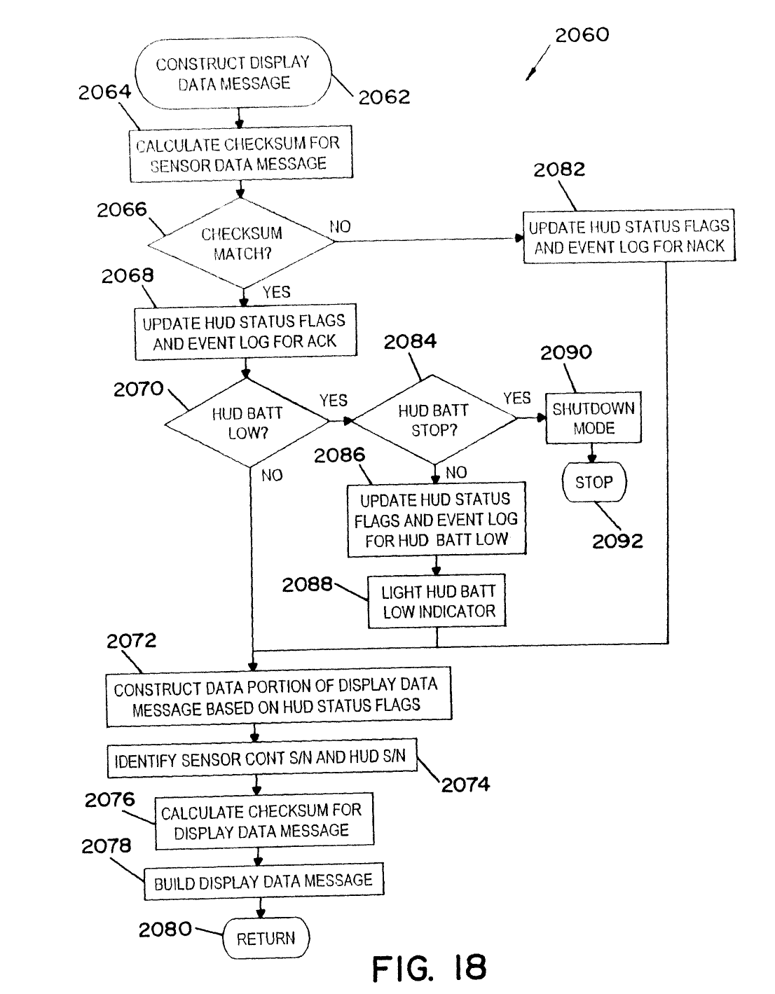

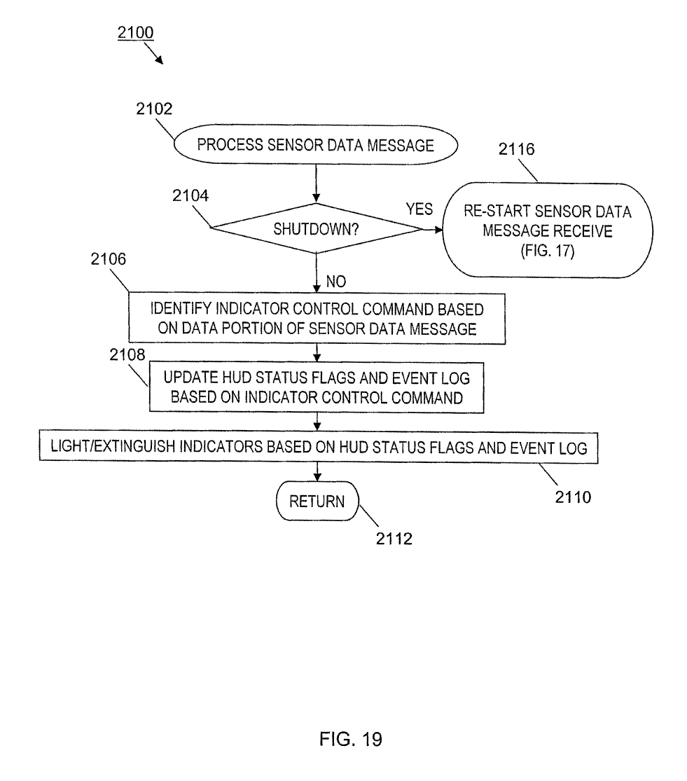

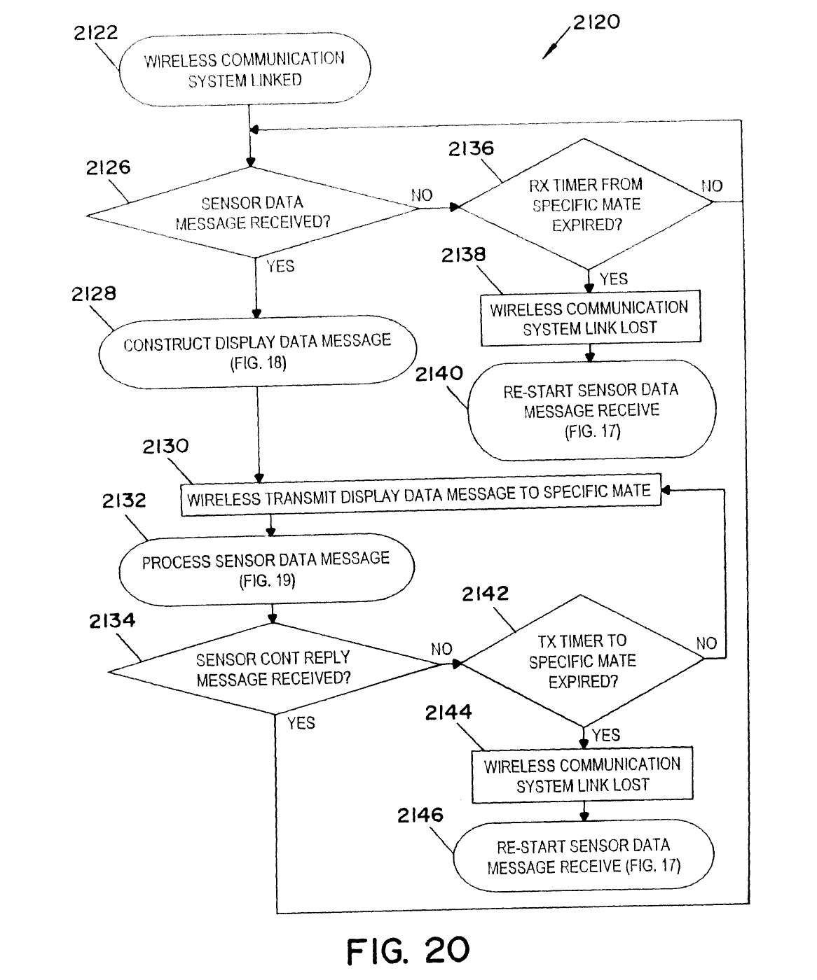

FIG. 17 is a flow chart of one non-limiting embodiment of a sensor data message receive process associated with a HUD assembly of an SCBA;

FIG. 18 is a flow chart of one non-limiting embodiment of a construct display data message process associated with a HUD assembly of an SCBA;

FIG. 19 is a flow chart of one non-limiting embodiment of a method for processing a sensor data message associated with a HUD assembly of an SCBA;

FIG. 20 is a flow chart of one non-limiting embodiment of a communication system linked process associated with a HUD assembly of an SCBA;

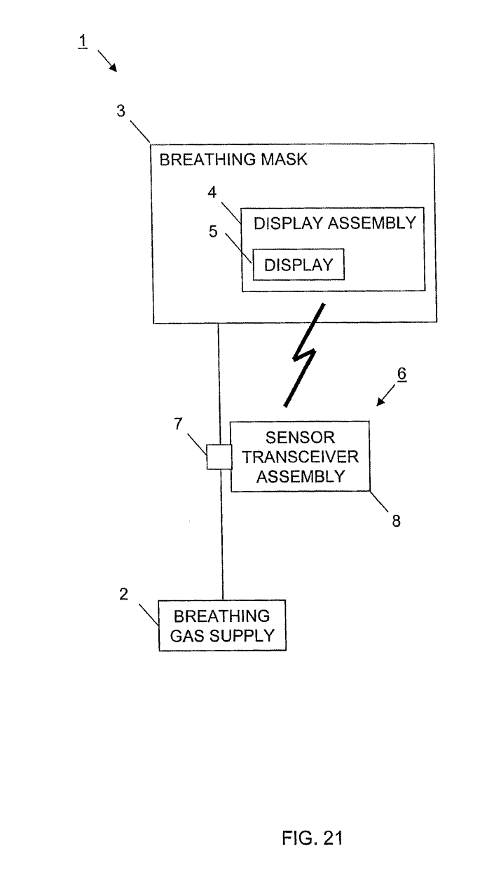

FIG. 21 is a block diagram of another exemplary embodiment of an SCBA;

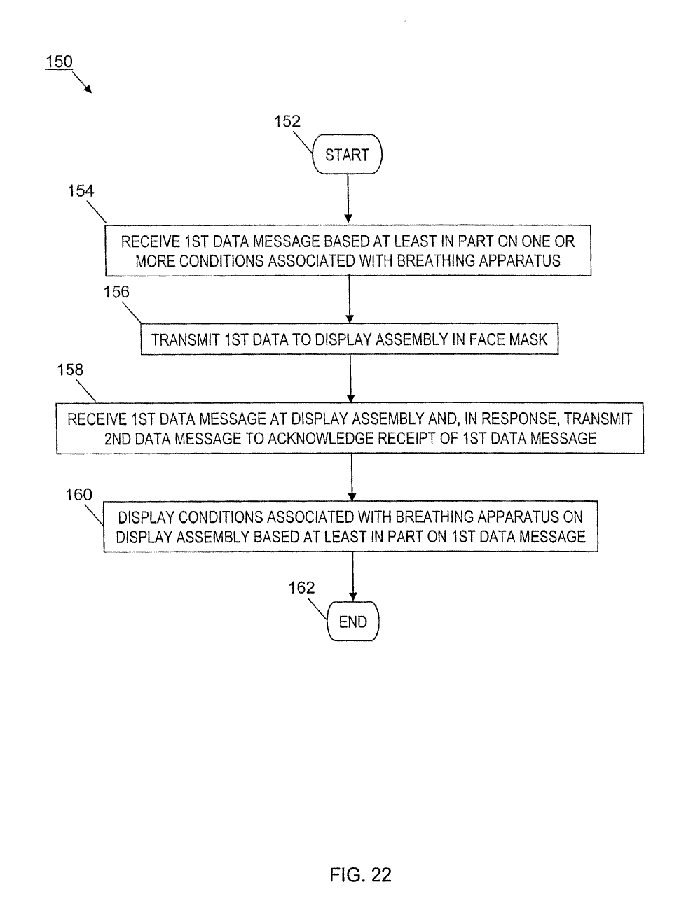

FIG. 22 is a flow chart of one non-limiting embodiment of a process for displaying one or more conditions associated with an SCBA to a wearer of the SCBA;

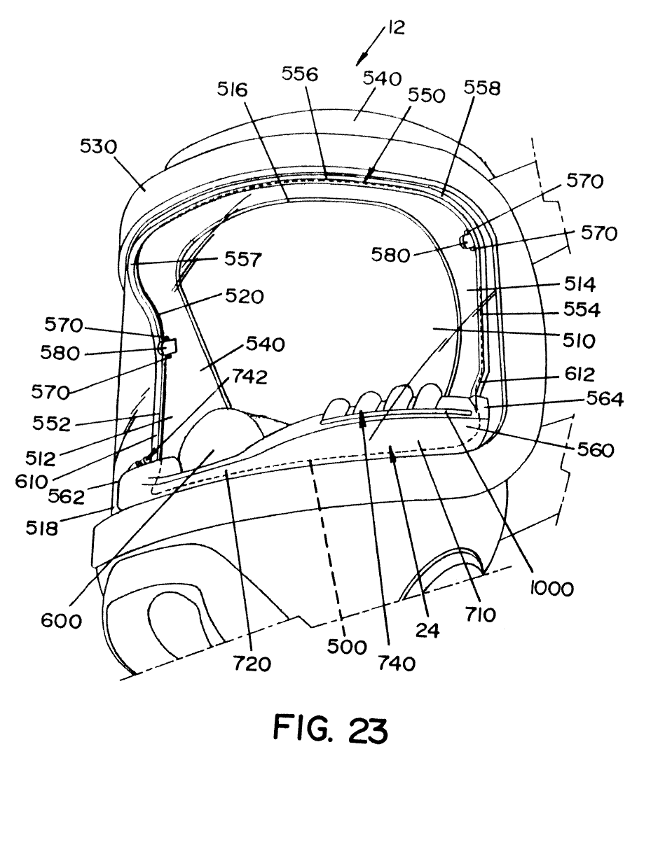

FIG. 23 illustrates one non-limiting view of a HUD for a SCBA in accordance with the present invention;

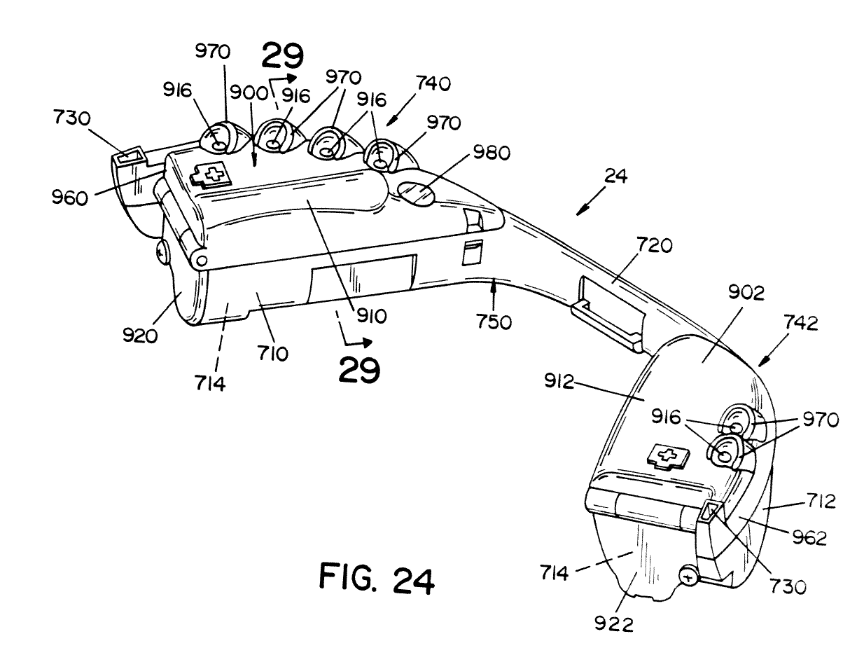

FIG. 24 is a perspective view of one non-limiting embodiment of a HUD in accordance with the present invention;

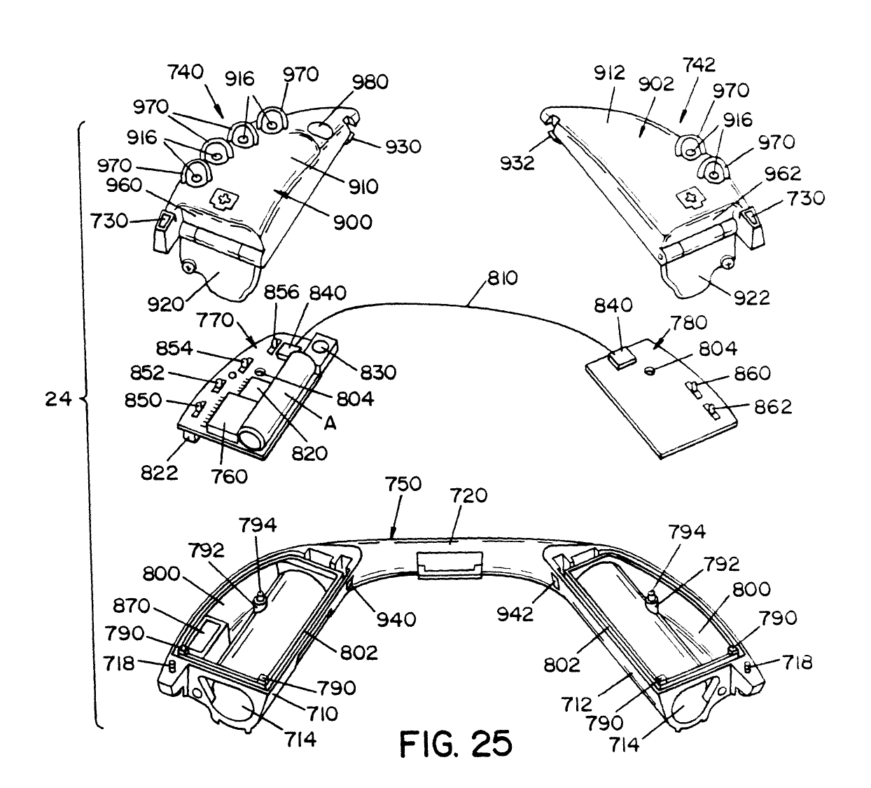

FIG. 25 is an exploded perspective view of the HUD of FIG. 24;

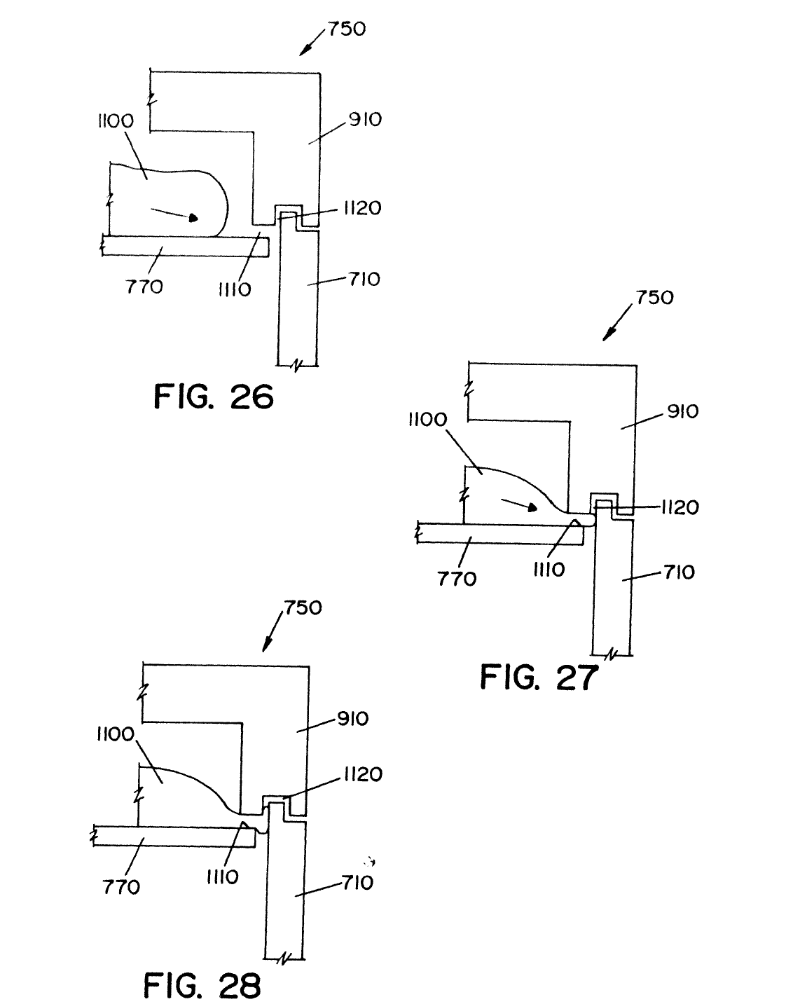

FIGS. 26-28 schematically illustrate one non-limiting embodiment of an arrangement for at least partially sealing an electronic housing.

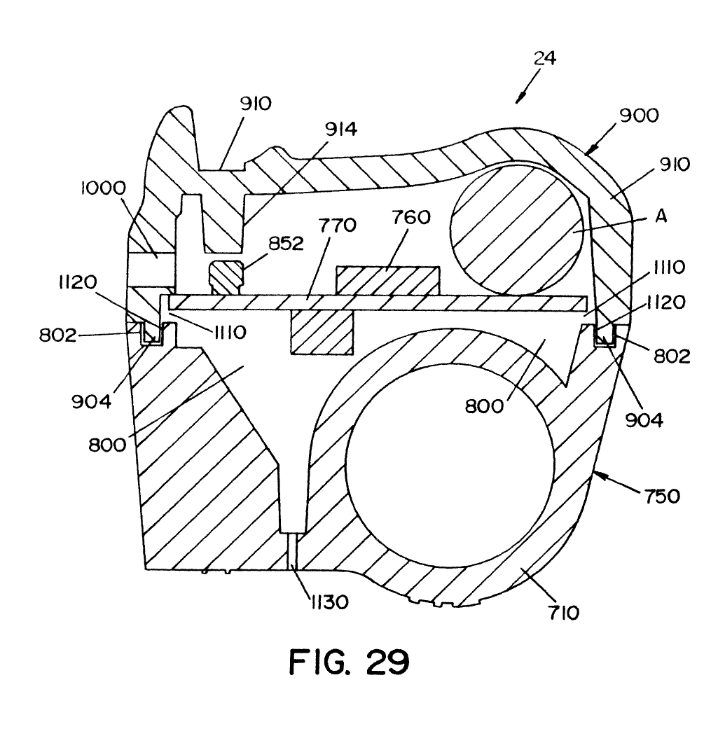

FIG. 29 is a cross-sectional view taken along line 29-29 in FIG. 24;

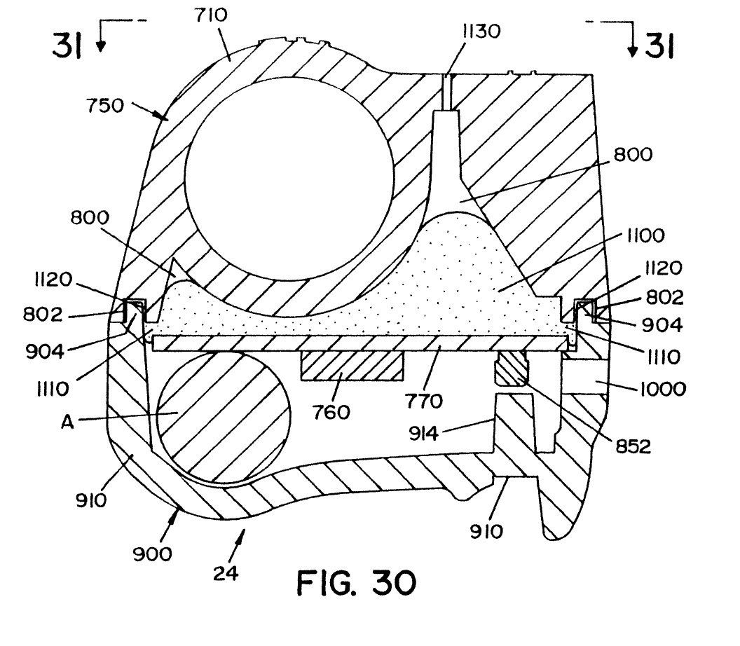

FIG. 30 is an 180 degrees rotated view of FIG. 29 illustrating application of a sealing material under the force of gravity; and,

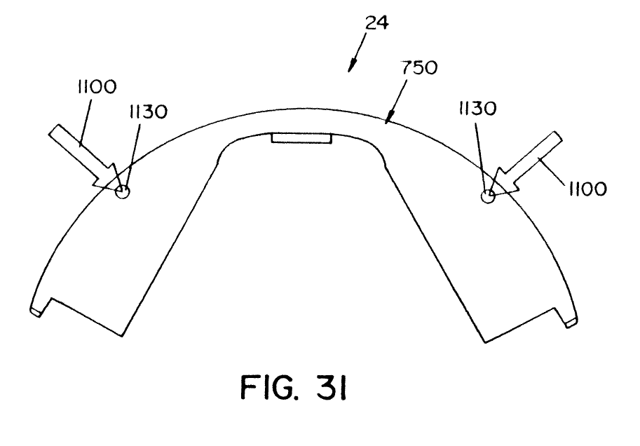

FIG. 31 is a cross-sectional view taken along line 31-31 in FIG. 30.

### DESCRIPTION OF INVENTION

Prior to discussing the various aspects of a wireless communications system for a HUD in an SCBA, a review of the definitions of some exemplary terms used throughout the disclosure is appropriate. Both singular and plural forms of all terms fall within each meaning.

“Controller,” as used herein, includes, hut is not limited, to any combination of devices, circuits, and/or logic that coordinates and/or controls the operation of one or more input and/or output devices. For example, a controller can include, but is not limited to, a device having one or more microcontrollers, microprocessors, and/or central processing units which may or may not be capable of being programmed to perform input and/or output functions.

“Data message,” as used herein, includes, but is not limited to, a communication by one or more signals that carry information, an ordered selection of an agreed set of symbols for the purpose of communicating some basic element of information, and/or a communication that provides information. For example, a data message can have some type of format and/or sequence that permits transmitting and/or receiving devices to interpret information exchanged via the message in the same manner; however, this is not required.

“Display assembly,” as used herein, includes, but is not limited to, an assembly that provides a visual representation of a received signal, a device that at least temporarily presents information in visual form, and/or a device for displaying data. For example, a display assembly can include, but is not limited to, one or more light emitting diodes (LEDs), liquid crystal displays (LCDs), incandescent lamps, and/or others types of visual indicators, either individually or in any combination.

“Electromagnetic field,” as used herein, includes, but is not limited to, a field of influence produced around a conductor by the current flowing through the conductor and having a) both electric and magnetic components, b) a combination of electric and magnetic fields of force, and/or c) a region in which electromagnetic radiation from a source exerts an influence on another object, with or without there being contact between them. The electromagnetic field includes a rapidly moving electric field and its associated magnetic field. An electric field is an invisible force field created by the attraction and repulsion of electrical charges and is measured in volts per meter. A magnetic field is an invisible force field created by a magnet and/or as a consequence of the movement of charges (i.e., flow of electricity). The magnetic field is perpendicular to both the electric lines of force and their direction.

“Electromagnetic wave,” as used herein, includes, but is not limited to, a) radiant energy with magnetic and electrical properties produced when electrical charges change their motion, b) radiant energy produced by oscillations of combined electric and magnetic fields, and/or c) disturbance, including vibrating electric and magnetic fields, that propagate outward from any electrical charge that oscillates and/or is accelerated. Electromagnetic waves include radio, infrared, visible light, and ultraviolet light waves, as well as microwaves, X rays, and gamma rays.

“Logic,” as used herein, includes, but is not limited to, hardware, firmware, software and/or combinations of each to perform a function(s), an action(s), and/or to cause a function and/or action from another component. For example, based on a desired application and/or needs, logic can include, but is not limited to, a software controlled microprocessor, discrete logic such as an application specific integrated circuit (ASIC), and/or other programmed logic device. Logic can also be fully embodied as software; however, this is not required.

“Signal,” includes, but is not limited to, one or more electrical signals, optical signals, electromagnetic signals, analog and/or digital signals, one or more computer instructions, a bit and/or bit stream, or the like.

“Software,” as used herein, includes, but is not limited to, one or more computer readable and/or executable instructions that can cause a computer and/or other electronic device to perform functions, actions, and/or behave in a desired manner. The instructions can be embodied in various forms such as, but not limited to, routines, algorithms, modules and/or programs including separate applications and/or code from dynamically linked libraries. Software can be implemented in various forms such as, but not limited to, a stand-alone program, a function call, a servlet, an applet, instructions stored in a memory, part of an operating system and/or other type of executable instructions. It will be appreciated by one of ordinary skill in the art that the form of software can be at least partially dependent on, for example, requirements of a desired application, the environment it runs on, and/or the desires of a designer/programmer or the like.

“Transceiver,” as used herein, includes, but is not limited to, a device that can both transmit and receive signals, a transmitter/receiver in a single package, and/or a combination of transmitting and receiving equipment in a common housing which may include some common circuit components for both transmitting and receiving. A “half duplex” transceiver can either transmit or receive. A “full duplex” transceiver can transmit and receive at the same time.

“Transmit,” as used herein, includes, but is not limited to, sending data from one point to another, sending a message or other information from one location to another, sending information over a communication line and/or a circuit, and/or sending out a signal by electromagnetic radiation (e.g., electromagnetic field and/or electromagnetic wave) and/or over a wire and/or cable (e.g., fiber optic cable, etc.).

“Wireless,” as used herein, includes, but is not limited to, describing a device that communicates with one or more other devices via electromagnetic radiation (e.g., electromagnetic field or electromagnetic wave) and/or communications that take place without the use of interconnecting wires and/or cables. Wireless devices can communicate even if they are not physically connected. Wireless can refer to lacking or not requiring wires and/or cables.

Referring now to the drawings wherein the showing is for the purpose of illustrating preferred non-limiting embodiments of the invention only and not for the purpose of limiting the same, as illustrated in FIGS. 1 and 21 there is shown two non-limiting embodiments of an SCBA 1 and 10 that are adapted to display one or more conditions associated with the SCBA 1 and 10 in accordance with the present invention.

Referring now to FIG. 21, SCBA 1 is shown to include a breathing gas supply 2 and a breathing mask 3 associated with the breathing gas supply 2. The breathing mask 3 includes at least one display assembly 4 that has one or more displays 5. The one or more displays 5 are designed to visually display one or more conditions of SCBA 1. As can be appreciated, the one or more displays 5 can also or alternatively be designed to provide audible sounds and/or vibrations to provide information about one or more conditions of SCBA 1. The SCBA 1 can also include a sensor assembly 6 which can include one or more sensors 7 for detecting and/or monitoring one or more conditions associated with the SCBA 1. The SCBA/can also include a sensor transceiver assembly 8 that is in communication with the display assembly 4. The sensor transceiver assembly 8 can be designed to a) receive one or more data messages that are at least partially based on the one or more conditions, and/or b) transmit the one or more data messages to the display assembly 4. The display assembly 4 which receives one or more data messages from the sensor transceiver can be designed to transmit one or more data messages to the sensor transceiver assembly 8 to acknowledge receipt of the one or more data messages transmitted from the sensor transceiver assembly to the display assembly; however, this is not required. The display assembly 4 can also be designed to display one or more conditions associated with SCBA 1 on one or more displays 5 based at/cast partially on the one or more data messages transmitted from the sensor transceiver assembly to the display assembly; however, this is not required.

With reference now to FIG. 1, another non-limiting embodiment of an SCBA 10 is illustrated. SCBA 10 includes a breathing mask 12, a sensor assembly 14, and a breathing gas supply 16. The breathing mask 12 is in fluidic communication with the breathing gas supply 16 when a valve 18 is opened. With valve 18 open, breathing gas can flow from the breathing gas supply 16, through the valve 18, to a first breathing hose 20, to the sensor assembly 14, to a second breathing hose 22, and then to the breathing mask 12. As can be appreciated, sensor assembly 14 can be located proximate to or integral with valve 18. In this arrangement, first and second breathing hose can be a single hose. FIG. 1A illustrates another non-limiting SCBA 10 which is similar to the SCBA disclosed in U.S. Pat. No. 7,089,930, entitled “Wireless Heads-Up Display for a Self-Contained Breathing Apparatus,” which is incorporated herein by reference in its entirety. As can be appreciated, other SCBAs such as, but not limited to the SCBAs disclosed in U.S. Provisional Application Ser. No. 60/772,452 filed Feb. 10, 2006, entitled “Heads-Up Display For A Self-Contained Breathing Apparatus,” which is incorporated herein in its entirety by reference, can be modified to include one or more of the features disclosed in the present invention.

With reference again to FIG. 1, breathing mask 12 can include a HUD assembly 24 to provide a wearer with a display indicating the status of parameters and/or conditions associated with the SCBA 10. The HUD assembly 24 can be wired and/or wirelessly connected to one or more other components of the SCBA 10. The sensor assembly 14 can include a pressure sensor 26 to sense a pressure level associated with the breathing gas and a sensor controller assembly 28. The sensor controller assembly 28 can be in operative communication with the pressure sensor 26 and the HUD assembly 24. As shown, communications between the sensor controller assembly 28 and the HUD assembly 24 can utilize any suitable form of wired and/or wireless communication. For example, in one non-limiting embodiment of the invention, the SCBA 10 includes a wireless communication system that can be compliant with the requirements of NFPA and/or other international standards.

The display in the HUD assembly 24 can be designed to indicate an amount of breathing gas remaining in the breathing gas supply 16 and/or other conditions associated with the SCBA 10. The sensor controller assembly 28 can be designed to send one or more sensor data messages to the HUD assembly 24 via wireless and/or wired communication, which one or more data messages can include one or more command to control operations of the HUD assembly 24, and/or other types of information (e.g., low power conditions for the sensor control, etc.). The HUD assembly 24 can be designed to send the HUD assembly one or more data messages to the sensor controller assembly 28 via wireless and/or wired communication in reply to acknowledge receipt of a one or more valid sensor data message; however, this is not required. When the HUD assembly is designed to send one or more data messages to the sensor controller in reply to acknowledge receipt of a one or more valid sensor data message, such sent one or more data messages by the HUD assembly can be used to provide notice of a problem associated with the sensor data message (e.g., non-acknowledgment, etc.), provide notice of low battery conditions associated with the HUD assembly 24, and/or other conditions associated with the HUD assembly 24. For example, FIG. 1 illustrates a sensor assembly 14 that can be used to sense the pressure level in breathing gas supply 16 and transmits a wireless signal to the HUD assembly 24 in mask 12 that is indicative of the amount of breathing gas in the breathing gas supply 16. As can be appreciated, the sensor assembly 14 can include a receiver and/or transceiver to received information from the HUD assembly 24 and/or from some location remote from the HUD assembly.

Referring now to FIGS. 1 and 2, there is illustrated one non-limiting embodiment of sensor assembly 14 that includes sensor controller assembly 28 and a pressure sensor 26 in accordance with the present invention. As illustrated in FIG. 1, the sensor assembly 14 includes a housing that can be affixed to one or more manifolds in the breathing gas hoses 20, 22. The one or more manifolds generally include one or more orifices to allow the one or more positions of the sensor assembly 14 to be inserted inline with breathing gas hoses 20, 22. Configured in this manner, the pressure of the breathing gas in breathing gas hoses 20, 22 can be sensed by pressure sensor 26. As can be appreciated, other or additional arrangements can be used to enable pressure sensor 26 to sense the pressure in breathing gas hoses 20, 22. As illustrated in FIG. 2, the sensor control assembly includes a sensor message construction logic 30, a transceiver assembly 32, and one or more batteries 33. The sensor control assembly can also include a communication port 36; however, this is not required. In one non-limiting arrangement, the sensor controller assembly 28 can be at least partially powered by one or more 9 volt batteries. As can be appreciated the sensor controller assembly 28 can be at least partially powered by one or more different batteries, additional types of batteries, and/or different or additional types of power sources (e.g., fuel cell, solar cell, etc.). As can also be appreciated, the one or more batteries and/or other types of power sources may or may not be rechargeable. As can also be appreciated, the one or more batteries and/or other types of power sources may or may not be replaceable by a user of the SCBA 10. As can also be appreciated, the sensor assembly 14 can include one or more visual indicators, audible indicators, and/or vibration indications to provide information regarding the power and/or charge level of one or more batteries and/or other types of power sources in the sensor assembly 14. As can also be appreciated, this power and/or charge level of one or more batteries and/or other types of power sources in the sensor assembly 14 can be transmitted to the HUD assembly 24 as illustrated in FIG. 1; however, this is not required.

As illustrated in FIG. 2, the transceiver assembly 32 can include a controller 46, a memory 48, a transceiver 50, an antenna 52, and a regulator 54; however, this is not required. As can be appreciated, the transceiver assembly 32 can include other or additional components (e.g., amplifier, etc.). In one non-limiting arrangement, the memory 48 can be embedded in the controller 46; however, this is not required. In another and/or alternative non-limiting arrangements, memory 48 can be partially or fully separate from controller 46. In another and/or alternative non-limiting arrangement, the regulator 54 can be used to provide voltage regulation and/or power distribution for one or more components of transceiver assembly 32 and/or sensor controller assembly 28; however, this is not required. In another and/or alternative non-limiting arrangement, regulator 54 can provide a relay and/or switching function to enable and/or disable power distribution to one or more components of the transceiver assembly 32 and/or sensor controller assembly 28. Further details of a transceiver assembly 32 that can be used in the present invention can be found in U.S. Pat. No. 7,089,930, which is incorporated herein by reference in its entirety.

The sensor controller assembly 28 is illustrated as including one or more batteries 33 that are in electrical communication with transceiver assembly 32 and regulator 54. As can be appreciated, the one or more batteries can be in electrical communication with one or more other or additional components of the sensor controller assembly 28. As can also be appreciated, one or more of the batteries can be located at a location remote to sensor controller assembly 28: however, this is not required. As can also be appreciated, a housing for the sensor controller assembly 28 can enable a user to recharge and/or replace one or more of batteries 33; however, this is not required. As can be appreciated, many types of batteries can be used (e.g., watch batteries, AAAA sized batteries, AAA sized batteries, custom sized and/or shaped batteries, etc.). The size, type, shape, voltage, etc. of one or more of the batteries for use in the SCBA will depend on several different factors (e.g., desired size and/or shape of one or more components of the SCBA, desired weight of the SCBA, the power requirements and/or power drain caused by one or more components of the SCBA, desired battery life for the one or more components of the SCBA, etc.). In one non-limiting arrangement, the one or more batteries used in the SCBA are designed to provide at least about 30-60 days of power to the SCBA when the batteries are fully charged. As can be appreciated, other minimal battery life times can be used. The sensor controller assembly 28 is illustrated as being in communication with pressure sensor 26. As can be appreciated many types of pressure sensors can be used. The pressure sensor 26 can be designed to generate an analog and/or digital signal. This signal can be used by the sensor controller assembly 28 and/or one or more other components of the SCBA to determine or interpret the amount of breathing gas remaining in the breathing gas supply 16. The pressure sensor 26 can be designed to communicate with sensor controller assembly 28 and/or one or more other components of SCBA via a digital signal and/or analog signal. The pressure sensor can be designed to be connected to sensor controller assembly 28 via a wire, cable and/or plug, or be wirelessly in commination with the sensor controller assembly 28. A sensor message construction logic 30 is illustrated as being included in the sensor controller assembly 28. The sensor message construction logic 30 is shown to be in communication with controller 46 and regulator 54; however, it can be appreciated that sensor message construction logic 30 can be in communication with other or additional components of the sensor controller assembly 28. A controller 46 is also illustrated as being included in the sensor controller assembly 28. The controller 46 is shown to be in communication with the communication port 36, transceiver 50, antenna 52, and regulator 54; however, it can be appreciated that controller 46 can be in communication with other or additional components of the sensor controller assembly 28. As illustrated in FIG. 1, sensor controller assembly 28 is also shown to include an antenna 52 that is in wireless communication W with the HUD assembly 24; however, it can be appreciated that sensor controller assembly 28 can be also or alternatively be in communication with the HUD assembly 24 via a wire and/or cable.

The memory 48 in controller 46 can be used to store a variety of information. For instance, memory 48 can be used to at least partially store software programs that can be executed by the controller 46. These software programs can control various aspects of operation of the transceiver assembly 32, sensor controller assembly 28, other components of the sensor assembly 14, and/or HUD assembly 24. The memory 48 can also or alternatively be used to store various parameters of one or more components of the transceiver assembly 32, sensor controller assembly 28, other components of the sensor assembly 14, and/or HUD assembly 24 (e.g., type, make, model of one or more components of the SCBA 10, service information of one or more components of the SCBA 10, length of use of one or more components of the SCBA 10, maintenance information of one or more components of the SCBA 10, presets and/or adjust parameters of one or more components of the SCBA 10, assembly and/or repair information of one or more components of the SCBA 10, etc.). The memory 48 can also or alternatively be used to store various types of records regarding the function and/or operation of one or more components of the SCBA 10 (e.g., current information and/or historical information of battery status, rate of battery use, estimate remaining time of battery power, current information and/or historical information geographic location of SCBA 10 [e.g., GPS information, LAN information, etc.], current information and/or historical information of pressure and/or sensor data, rate of use air consumption from breathing gas supply, estimate remaining air from breathing gas supply, current time, time correlation and/or geographic correlation to one or more types of data stored in memory 48, etc.). When time information is stored in memory 48, the time information can be actual time and/or actual date information, and/or can be time relative to when one or more components of the SCBA have been activated and/or deactivated. Memory 48 can be a variety of different types of memory (e.g., RAM, ROM, flash memory, etc.). In one non-limiting arrangement, at least a portion of memory 48 includes flash memory and/or some other type of memory that can be reprogrammed using a programming device connected to one or more communication ports 36; however, this is not required. The one or more communication ports 36 can be designed to accept one or more types of cables (e.g., fire wire, USB, serial cable, phone cable, ethernet cable, etc.). The programming device can be any number of devices such as, but not limited to, a portable computer (e.g., laptop computer, notebook computer, tablet computer, PDA device, Palm PC device, Blackberry device, etc.), desktop and/or mainframe computer, cell phone, etc. As illustrated in FIG. 2, communication port 36 allows for communication with controller 48; however, it can be appreciated that communication port 36 can be used to allow for communication with other or additional components of SCBA 10.

Referring now to FIGS. 1 and 3, the display logic 62 of HUD assembly 24 that is at least partially is located in breathing mask 12 can include a controller 66, a memory 68, an antenna 70, a transceiver 74, one or more indicators 76, a light sensor 78, a power monitoring circuit 80, and/or a communications port 82. As can be appreciated, display logic 62 can include other or additional components. As can also be appreciated, one or more components of display logic 62 can located remotely from breathing mask 12. As illustrated in FIG. 3, memory 68 can be embedded within the controller 66; however, this is not required. It can be appreciated that all or part of the information in memory 68 can also be located in controller 66 or vise versa. As can also be appreciated, all or a portion of memory 68 can be included with memory 48 as previously described above with regard to FIG. 2. Likewise, when memory 68 is used, such memory can be the same or similar to the type and/or function of memory 48 as previously described. The types of information included in memory 68 can be one or more types of information as described above that can be included in memory 48. As can be appreciated, all or part of memory 68 can be substituted for memory 48 or vice versa.

The one or more indicators 76 of display logic 62 can be many types of indicator(s). For instance, one or more indicators can be a sound indicator, a visual indicator (LED light, LCD light or panel, fluorescent, light, incandescent light, etc.), etc. In one non-limiting arrangement, one or more of the indicators are LED indicators. The number of indicators and/or type of indicators is non-limiting. For example, the mask can be designed to include six (6) LEDs, four LEDs located on the user's left side and two LEDs on the user's right side. This non-limiting arrangement is illustrated in FIG. 23. As can be appreciated, a mask that includes a smaller or a greater number of LEDs can be used (e.g., See SCBA having four LEDs in U.S. Pat. No. 7,089,930, which is incorporated herein by reference in its entirety, etc.).

Referring again to FIG. 23, the mask 12 includes a generally transparent protective shield 510, an inner rim 520 disposed at least partially around a periphery of the protective shield inside the mask, and an outer rim 530 disposed at least partially around the shield outside the mask. The generally transparent protective shield 510 is designed to enable a user of the mask to have a clear field of vision; however, this is not required. The HUD assembly 24 that is at least partially included in the mask can be designed to fit in a wide variety of different masks. For example, a variety of existing SCBA masks with a protective shield and a rim are commercially available which HUD assembly 24 could be configures for use therein. The HUD assembly 24 is generally located in the mask 12 so as to not interfere with the mask's breathing function through port 600 or the field of view characteristic through the shield 510. The HUD assembly 24 is generally located so as to be in the field of view of a user of the mask without limiting the user's field of view outside through shield 510 when the user is wearing the mask; however, this is not required. The rim 520 of the shield 510 can be designed to provide a stepped surface at least partially around the periphery of the protective shield. Rim 520 can be designed to connect to a mask portion 540 of the mask 12. Portion 540 can be designed to at least partially form a seal against a portion of the user's face when the mask is worn by the user such that mask portion 540 and protective shield 510 at least partially form a sealed environment at the front portion of a user's face. A top section and a side section of mask portion 540 is illustrated schematically in phantom lines to simplify the drawing. As previously mentioned, the oral-nasal breathing port 600 is provided inside the mask to enable a user to breathe air from the breathing gas supply 16 when the mask is worn by the user. As illustrated in FIGS. 1, 1A, and 2, the mask 12 is in fluid communication with a breathing gas supply 16 via breathing hoses 20, 22 and valve 26. The breathing gas supply 16 can be a SCBA breathing gas tank; however, this is not required. All or a portion of the sensor assembly 14 can be disposed on and/or in the breathing hoses 20, 22. The mask 12 generally includes one or more straps used to secure the mask to the head of the user; however, this is not required.

As illustrated in FIG. 23, an elongated retaining member 550 can be used to connect to first and second end portions 562, 564 of housing 560 of HUD assembly 24. The retaining member 550 can be designed to engage the rim 520 around the periphery of the protective shield 510 inside the mask so as to at least partially secure display housing 560 to the mask. The retaining member 550 can be secured to the HUD assembly 24 in a variety of ways (e.g., screw, snap-lock engagement, adhesive, weld or melt connection, screw, rivet or other type of mechanical connection, etc.). The retaining member 550 includes first and second leg portions 552, 554 connected by a center portion 556. The leg portions 552, 554 are generally configured to conform to the shape of side portions 512, 514 of the shield 510; however, this is not required. The center portion 556 of the retaining member is generally configured to conform to the shape of a top portion 516 of the shield 510; however, this is not required. Curved transition areas 557, 558 of the retaining member connect the leg portions 552, 554 to the center portion 556. These curved portions are also generally configured to conform to the shape of the shield 510; however, this is not required. The center portion of the retaining member 556 is illustrated as being generally curved to conform to the contour of the shield 510; however, it will be appreciated that other shapes for the center portion and/or other portions of the retaining member can be used. The various portions of the retaining member can be initially formed in a generally straight configuration or some other initial configuration and then be subsequently bent to conform to the shield contour when installed; however, this is not required. The retaining member 550 can include one or more connection members to secure the retaining member to the mask; however, this is not required. As illustrated in FIG. 23, the retaining member includes a pair of spaced apart tab engaging protrusions 570. The protrusions 570 are generally configured for positioning on opposite sides of tabs 580 that extend behind the rim 520 inside the mask 12 so as to at least partially inhibit movement of the retaining member 550. As can be appreciated, other or additional arrangements can be used to secure the retaining member to the mask. The retaining member can be made from a wide variety of different materials such as, but not limited to, metal, plastic, rubber, fiber and/or carbon reinforced material, etc. Some non-limiting specific examples of materials that can be included in the retaining member are molded plastic, metal wire (e.g., shaped and/or tempered spring wire, etc.). As mentioned above, the retaining member 550 can be coupled to the housing 560 of the HUD assembly 24 in any known manner. For instance, the ends 610, 612 of the leg portions 552, 554 of the retaining member can be disposed in recesses in the housing 560 of the HUD assembly 24 to couple the retaining member 550 to the HUD assembly 24; however, this is not required.

Referring now to FIGS. 23 and 24, the contour of the housing 560 of the HUD assembly 24 is generally designed to substantially match the contour of the lower portion 518 of the protective shield 510; however, this is not required. The HUD assembly 24 is at least partially positioned in the mask 12 such that the HUD assembly is at least partially supported by a lower portion 500 of the rim 520; however, this is not required. The HUD assembly can also be designed to at least partially engage the lower portion 518 of the protective shield 510; however, this is not required. The battery compartment portions 710, 712 of the HUD assembly are disposed on opposite sides of the breathing port 600; however, this is not required. As can be appreciated, a single battery compartment or more than two battery compartments can be included in the HUD assembly. As can also be appreciated, the power supply for the HUD assembly can be positioned remotely from the HUD assembly. As illustrated in FIG. 24, a connecting portion 720 spans the breathing port 600. Ends 610, 612 of the leg portions of the retaining member 550 can be inserted into the mounting recesses 730 in the HUD assembly 24; however, this is not required. The center portion 720 of the retaining member 550 can be designed to be initially pulled down to allow the leg portions 552, 554 to be positioned against the shield 510 and the rim 520 with the pairs of tab engaging protrusions 570 disposed around the tabs 580; however, this is not required. Once the leg portions 552, 554 are in position, the center portion 720 can be moved against rim 520 and the upper portion 516 of the shield 510; however, this is not required. As can be appreciated, there are many other or additional ways that the retaining member can be positioned in the mask. In one non-limiting arrangement, the center portion 720 snaps can be snapped into place against the rim 520 and the shield 510; however, this is not required. The engagement between the retaining member 550 and the rim 520 can be designed to at least partially press the HUD assembly 24 against the shield 510 and the rim 520 to at least partially secure the HUB assembly 24 in the mask; however, this is not required. Although the use of a retaining member 550 has been described in detail regarding the securing of the HUD assembly in mask 12, it will be appreciated that many other of additional arrangements can be used to at least partially secure the HUD assembly in the mask (e.g., adhesive, Velcro, screws, rivets, snaps, latches, tongue and groove connectors, hooks, etc.).

Referring now to FIG. 24, the HUD assembly 24 includes a displays 740, 742 that are designed to provide information to the user of the mask. As previously mentioned above, such information can include the condition and/or status of one or more components of the SCBA 10. For instance, displays 740, 742 can be used to provide information regarding 1) the amount of breathing gas remaining in the breathing gas supply, 2) the battery power level of the HUD assembly and/or the sensor assembly, 3) the time of day, 4) the time of use by the user of the mask, 5) location information, 6) information about other people in a certain area, 7) map information, and/or the like. In one non-limiting arrangement, one display 740 provides information regarding the amount of breathing gas remaining in the breathing gas supply 16 and at least one other display 742 provides information regarding the battery status of the HUD assembly 24 and/or the sensor assembly 14. As can be appreciated, the amount of breathing gas remaining and the battery status are merely two non-limiting examples of the wide variety of parameters that can be displayed by the HUD assembly. As can also be appreciated, HUD assembly does not need to be designed to display both the battery status information and information about the amount of gas remaining in the breathing gas supply. The HUD assembly 24 can be designed to display any desired parameter (e.g., information about the user, information about the user's environment, the condition of user's equipment and/or tools, etc.). The HUD assembly can be hardwired to sensors that sense displayed parameters and/or the HUD system can be wirelessly coupled to a transmitter and/or transceiver that transmits information from one or more sensors. As can be appreciated, the HUD assembly can be hardwired to sensors and/or wirelessly communicate one or more sensors to send information to and/or receive information from one or more sensors.

Referring now to FIGS. 24 and 25, the HUD assembly 24 includes a housing 750, one or more displays 740, 742, and a receiver 760. As previously discussed, housing 750 includes a first battery compartment portion 710 and a second battery compartment portion 712 that are connected by a connecting portion 720. Each battery compartment portion includes a compartment 714 within which a battery (e.g., AA battery, AAA battery, etc.) for powering at least a portion of HUD assembly 24 resides. The first battery compartment portion 710 and the second battery compartment portion 712 are designed to be positioned on opposite sides of the breathing port 600 when the housing 750 is installed in the mask; however, this is not required. For example, one or more battery compartments can reside in the same side of the housing.

As illustrated in FIG. 25, there are provided first and second circuit boards 770, 780 that are mounted on the first and second battery compartment portions 710, 712. The circuit boards 770, 780 can be assembled with the housing in a wide variety of different ways. As can be appreciated, only one circuit board can be used, or more than one circuit board can be included in one or both battery compartments. As can also be appreciated, one or more circuit boards can be located in other or additional regions on housing 750. In one non-limiting arrangement, the circuit boards 770, 780 can be supported on the housing 750 by standoffs 790, 792. A hole 804 is disposed in each of the circuit boards 770, 780 to accept a pin portion 794 of standoff 792 to align the circuit boards with the housing 750. As can be appreciated, many other or additional arrangements can be used to align one or more of the circuit boards in the housing. The standoffs 790, 792 can be used to create a gap between the circuit boards 770, 780 and an inner surface of the housing; however, this is not required. A cavity 800 is defined in the compartment portions 710, 712 behind each of the circuit boards 770, 780 when the circuit boards are positioned in the compartment portions. The circuit boards 770, 780 and the one or more batteries can be interconnected by one or more conductors 810. The conductors 810 can be designed to form one or more electrical connections between the battery compartment portions 710, 720 through the connecting portion 720 when the HUD assembly 24 is assembled; however, this is not required. In one non-limiting arrangement, electronic components of the HUD assembly 24 such as, but not limited to, LEDs 850, 852, 854, 856, 860, 862, a controller 820, alight sensor 830, a connector 840 and/or an antenna A can be mounted to one or more of the circuit boards, as can be appreciated other or additional components can be connected to the one or more of the circuit boards (e.g., filter, surge protector, etc.). As illustrated in FIG. 24, the LEDs are surface mounted to the printed circuit board; however, this is not required.

As illustrated in FIGS. 23-25, display 740 includes four (4) LEDs 850, 852, 854, 856 mounted on the printed circuit board 770. As can be appreciated, a different number of LEDs and/or a different type of display can be included on circuit board 770. As will be described in more detail below, LEDs 850, 852, 854, 856 can be used to as a pressure display to represent various levels of breathing gas in the breathing gas supply. The pressure information can be conveyed to a user by the display using a combination of LED color and/or position. The LEDs can be arranged in any orientation including horizontal, vertical, or any oblique angle, and/or can include a variety of shapes and/or sizes depending on the space constraints. Additionally, or alternatively, discrete LEDs or a bank or array of LEDs can be employed. As can be appreciated, other or additional display configurations can be used (e.g., backlit Liquid Crystal Displays (LCDs), incandescent lamps, etc.). As can be appreciated, any combination of LED color, brightness, and position can be employed to provide a status indication regarding pressure information or some other or additional information. In one non-limiting arrangement, LEDs 850 and 852 can be green in color when illuminated, LED 854 can be yellow when illuminated, and LED 856 can be red when illuminated. In an alternative arrangement, LED 850 can be green when lit, LED 852 can be yellow when lit, LED 854 can be amber when lit, and LED 856 can be red when lit. As can be appreciated, other color arrangements can be used. In one non-limiting arrangement, when the amount of breathing gas in the breathing gas supply 16 is greater than ¾ full, all four LEDs (850, 852, 854, 856) can be illuminated; however, this is not required. When the amount of breathing gas in the breathing gas supply 16 is less than ¾ and greater than ½ full, three LEDs (852, 854, 856) can be illuminated; however, this is not required. When the amount of breathing gas in the breathing gas supply 16 is less than ½ and greater than ¼, two LEDs (854, 856) can be illuminated; however, this is not required. When the amount of breathing gas in the breathing gas supply 16 is less than ¼, one LED (856) can be illuminated; however, this is not required. As can be appreciated, other lighting sequences can be used to indicate the amount of breathing gas in the breathing gas supply. As can also be appreciated, other percentages of air in the breathing gas supply 16 can cause one or more of the LEDs to be lit. As can also be appreciated, one LED can light at a time to indicate a certain gas supply level. As can be appreciated, other types of visual indicators can be used (e.g., light bar, digital display to indicate percentage values, graph display, etc.). As can also be appreciated, other indicators (e.g., audible indicators, vibration indicators, etc.) can be included in the mask and/or on the HUD assembly to notify a user of a certain gas supply level and/or other type of condition. As can also be appreciated, LEDs that have multiple colors can be used. For example, a LED that can display 2 or more colors can be used in place of one or more LEDs that only displays a single color.

Display 742 includes two LEDs 860, 862. As can be appreciated, only one LED or more than two LEDs can be included on circuit board 780. In one non-limiting arrangement, LED 862 can be a battery status display that represents that a battery level of the HUD assembly 24 has fallen below a threshold value when the LED is illuminated; however, this is not required. As such, LED 862 can be designed to illuminate when the receiver battery status and/or the transmitter/transceiver battery status in the HUD assembly and/or one or more other battery sources in the SCBA indicate that the voltage output of the battery has fallen below a predetermined minimum voltage and/or energy level; however, this is not required. LED 860 on circuit board 780 can be a programmable auxiliary LED; however, this is not required. This auxiliary LED 860 can be used to provide an alert from a personal alert safety system, commonly referred to as PASS; however, this is not required. A controller 820 disposed on one or both of the circuit boards can be programmed via an input header 822 mounted on the printed circuit board; however, this is not required. The input header 822 can include a communications port for connection to a programming device such as, but not limited to a computer, PDA, etc; however, this is not required. As illustrated in FIG. 25, housing 750 includes a header access opening 870 that allows the controller 820 to be programmed when the printed circuit board 770 is installed in the housing 750. This programming allows the auxiliary LED 860 to be programmed to represent any parameter selected by the user. As can be appreciated, none or the LED can be programmable LED, or more than one LED on circuit boards 770, 780 can be programmable LEDs that can be at least partially programmed by one or more controllers using one or more input headers 822. Such programmable LED, when used, allows a user to at least partially customize the display 740, 742.

In another non-limiting arrangement, the group of two LEDs 860, 862 can include a red LED 862 and a yellow LED 860; however, this is not required. The red LED 860 can be used to provide pre-alert and/or alarm indication that can be associated with a personal alert safety system (PASS); however, this is not required. The yellow LED 862 can be used to indicate a low power source for the HUD assembly 24 and/or the sensor controller assembly 28; however, this is not required. As can be appreciated, one or both circuit boards can include a different arrangement of displays 740, 742, a greater or lesser number of LEDs, etc., different or additional types of LEDs, etc., different or additional colors of LEDs, etc., and/or LEDs, etc. that are capable of being selectively illuminated in one or more of multiple colors; however, this is not required.

Referring now to FIGS. 24 and 25, housing 750 includes first and second removable covers 900, 902 that are designed to be connectable to the battery compartment portions 710, 712, respectively. The first and second removable covers 900, 902 are designed to at least partially seal the housing 750, allow the one or more batteries in battery compartment portions 710, 712 to be accessed, and/or include a plurality of windows 916 that allow displays 740, 742 that include LEDs 850, 852, 854, 856, 860, 862 to be at least partially viewed inside the mask 12. Covers 910, 912 each include a printed circuit board covering portion 910, 912, respectively, and a battery door 920, 922, respectively; however, this is not required. The covers can include battery doors 920, 922 that are generally hingedly connected to each covers 910, 912; however, this is not required. The battery doors 920, 922 can be designed to at least partially close the battery compartment 714 and/or secure the rear end 960 of the cover portion to the battery compartment portions 710, 712. The battery doors 920, 922 can include a latch or other type of arrangement that engages a latch tab or the like on the housing 750 to facilitate in securing the battery doors in a closed position; however, this is not required. The printed circuit board covering portions 900, 902 are generally sized to fit over at least a portion of the printed circuit boards 770, 780 and to mate with the compartment portions 710, 712; however, this is not required. One or both of the covering portions 900, 902 can include a protrusion around its periphery 904 (See FIGS. 29-30) that fits within a recess 802 that extends around the periphery of the compartment portions 710, 712 to facilitate in the formation of a seal between one or both of the covering portions 900, 902 and the compartment portions 710, 712; however, this is not required. In another non-liming arrangement, one or both of the coverings 900, 902 portions can include a recess around their periphery that accepts a protrusion that extends around the periphery of one or both the compartment portions to facilitate in the formation of a seal between one or both the covering portions 900, 902 and the compartment portions 710, 712; however, this is not required. A latch member 930, 932 as illustrated in FIG. 25 can be provided to extend from each of the covering portions 900, 902; however, this is not required. The latch members 930, 932 are designed to cooperate with a latch recess 940, 942, respectively in the corresponding compartment portion 710, 712 to at least partially secure a forward end of each covering portion 740, 742 to the corresponding compartment portion 710, 712. On or more screw 718 can be used to engage a recess in covering portions 900, 902 so as to facilitate in securing and maintaining the covering portions 900, 902 in position relative to compartment portions 710, 712; however, this is not required. A mounting recess 730 is defined in a rear end 960, 962 of each of the covering portions 900, 902. The mounting recesses 730 allow ends 610, 612 of the legs 552, 554 of the retaining member 550 to be coupled to the HUD assembly housing 750. The mounting recesses 730 can be replaced with any coupling arrangement that allows the retaining member 550 to be connected to the HUD assembly housing 750. The covering portions 900, 902 define a concave surface that fits over the components mounted on the printed circuit boards 770, 780. As can be appreciated, the covering portions can have other types of shaped surfaces. One or both printed circuit boards 770, 780 can be at least partially recessed in the compartment portions 710, 712 to accommodate the components mounted on the printed circuit boards; however, this is not required. One or more portions of the covering portions 900, 902 and/or component portions 710, 712 can be formed of a non-transparent material, a semi-transparent material or a transparent material. As can also be appreciated, one or more portions of housing 750 can be formed of a non-transparent material, a semi-transparent material, colored material or a transparent material. One or more of the covering portions 900, 902 and/or component portions 710, 712 can include a latch and/or locking arrangement to at least partially secure the covering portions 900, 902 in a closed position on the component portions 710, 712; however, this is not required. When a latch and/or locking arrangement is used, such arrangement is generally a mechanical arrangement; however, this is not required.

Referring again to FIGS. 24 and 25, windows 916 are typically included on covering portions 900, 902; however, this is not required. One or more of windows 916 can include a shroud portion 970 that is positioned at least partially around one or more windows. The shroud portions can be used to at least partially block light emitted by one or more of the LEDs 850, 852, 854, 856, 860, 862 from being emitted toward shield 510; however, this is not required. As such, the one or more shroud portions can be used to at least partially prevent the light from one or more of the LEDs from being reflected by the shield 510, which reflection of light could interfere with the user's vision. As can be appreciated, one or more of the shroud portions can be eliminated from one or both of the covering portions 900, 902. One or more of the windows 916 can include a light pipe 914 (See FIGS. 29-30) that at least partially extends from covering portions 900, 902; however, this is not required. When one or more light pipes 914 are used, such light pipes can be designed to terminate a short distance from or contact with one or more LEDs, etc. on the circuit boards. The light pipes can be used to at least partially direct light from one or more LEDs, etc. and/or at least partially out of one or more windows 916 and into the view of the user; however, this is not required. The light pipes, when used, are generally formed of a transparent material. One acceptable transparent material is LEXAN, available from General Electric.

One or more of the cover portions can also include a light sensor window 980; however, this is not required. The light sensor window 980, when used, allows ambient light to be sensed by an ambient light sensor 830 mounted on the printed circuit board 770 for purposes of controlling the illumination intensity of one or more LEDs 850, 852, 854, 856, 860, 862.

Referring now to FIG. 23-25, the HUD assembly can include one or more windows 916 that allow one or more LEDs 850, 852, 854, 856, 860, 862 to be viewed by the user when the mask is being worn by the user, and also include one or more windows 1000 that allow one or more of the LEDs 850, 852, 854, 856, 860, 862 to be viewed through protective shield 510; however, this is not required. The one or more windows 1000 in combination with the one or more LEDs 850, 852, 854, 856, 860, 862 can be used to enable one or more people that are not wearing mask 12 to see the status of one or more parameters indicated by the one or more LEDs 850, 852, 854, 856, 860, 862. As a result, a person other than the user of the mask can look out for the person wearing the mask 12. For instance, the one or more windows 1000 can enable another person to determine the amount of breathing gas remaining for the user wearing the mask 12 and inform the user of such status and/or rescue the user based on the information identified in one or more of the windows 1000; however, this is not required. As illustrate in FIG. 23, the HUD assembly can include a single window 1000 that is large enough to view all four of the LEDs 850, 852, 854, 856. As can be appreciated, the HUD assembly can include another single window 1000 that is large enough to view all the two LEDs 860, 862. As can be appreciated, the HUD assembly can include a single window 1000 that is large enough to view all six LEDs 850, 852, 854, 856, 860, 862. As can also be appreciated, the HUD assembly can include individual windows for one or more of the LEDs. As can also be appreciated, one or more light pipes 914 can be used to facilitate in light transmission form one or more LCDs through one or more of the windows 1000; however, this is not required.

Referring now to FIGS. 26-28, there is illustrated one non-limiting arrangement to at least partially form a seal between one or more components of the SCBA 10. Specific reference will be made to at least partially forming a seal in housing 750 of the HUD assembly 24; however, it will be appreciated that such sealing arrangement can be used on other components of the SCBA 10 such as, but not limited to pressure sensor 26, sensor assembly 14, etc. As illustrated in FIGS. 26-28, there is shown a portion of component portion 710 and covering portion 910. A seal is formed between component portion 710 and covering portion 910 by use of a sealing material 1100. As illustrated in FIG. 26, the sealing material 1100 is initially applied to circuit board 770. As illustrated in FIG. 27, a portion of the sealing material 1100 flows and/or is at least partially forced into at least a portion of gap 1110 that exists between covering portion 910 and circuit board 770. The sealing material 1100 can be at least partially forced into gap 1110 in a wide variety of different ways. For example, the sealing material 1110 can be injected under pressure into a portion of housing 750 to force the sealing material 1100 into any available space within the housing, including gap 1110 as illustrated in FIG. 27; however, this is not required. In another and/or alternative arrangement, the weight of the sealing material 1100 that is applied to the interior of the housing 750 can cause a portion of the sealing material to flow toward and at least partially fill gap 1110. As illustrated in FIGS. 27 and 28, the sealing material first flows to gap 1110 formed between the circuit board 770 and covering portion 910. Thereafter, the sealing material continues to flow into gap 1120 that is formed between covering portion 910 and component portion 710 to form a seal therebetween. As shown in FIGS. 26-28, the sealing material 1100 is designed and formulated to at least partially fill and seal gaps 1110 and 1120. The sealing method and arrangement allows for a seal to be formed inside housing 750, without having to cover both sides of the circuit board 770. As illustrated in FIG. 28, the sealing material 1100 is located primarily on the top surface of the circuit board. Little, if any, sealing material 1100 is located on the bottom surface of the circuit board. As mentioned above, the sealing arrangement illustrated in FIGS. 26-28 can be used in other areas of housing 750 of HUD assembly 24 and/or can be used to form a seal in other component of the SCBA 10.

Referring now to FIGS. 25, 29-31, there is illustrated an arrangement and method for at least partially forming a seal between the covers 900, 902 and the battery compartment portions 710, 712. FIGS. 29 and 30 make specific reference to the forming of a seal between cover 900 and battery compartment portion 710; however, it will be appreciated that the same or similar arrangement and the method for forming a seal can be accomplished between cover 902 and battery compartment portion 712 and/or other portions of housing 750 and/or other components of SCBA 10. As illustrated in FIG. 25, 29-31, the printed circuit board 770 is positioned on standoffs 790, 792 that provide a gap 1100 between the printed circuit board 770 and the battery compartment portions 710, 712. Cover 900 can be secured to the housing 750 as discussed above. As illustrated in FIGS. 29 and 30, LED 852 of display 740 of HUD assembly 24 is oriented such that the battery compartment portion 710 is positioned above the printed circuit board 770. A sealing material 1100 is used to at least partially fill a void or cavity 800 between the printed circuit board 770 and the battery compartment portion 900. One of more openings 1130 in the base of housing 750 are provided to allow sealing material 1100 to be provided to cavity 800. The sealing material 1100 can be injected as indicated by the arrows in FIG. 31 through one or more of the openings 1130 in housing 750. The sealing material 1100 then flows into cavity 800 and ultimately contact a bottom portion of circuit board 770. When the housing in positioned upside down or in some other non-upright position, the force of gravity can be used to at least partially cause the sealing material 1110 to flow through opening(s) 1130 and into cavity 800. The pressure applied on the sealing material 1110 as it is inserted into the housing 750 and/or the force of gravity on the sealing material can be used to cause at least a portion of the sealing material to flow toward and into gaps 1110 and 1120 so as to form a seal between the battery compartment portion 710 and cover 900. A seal can also be formed between the circuit board 770 and the compartment portion 710 and/or cover 900; however, this is not required. As illustrated in FIG. 30, little, if any, sealing material flows onto the top side of circuit board 770. As such, the sealing material does not partially or fully cover any of the LEDs on the top side of the circuit board and/or partially or fully cover any other components on the top side of the circuit board, and/or the light pipes, if used, on the underside of cover 900. As can be appreciated, the sealing material can be used to at least partially secure together the compartment portion 710 and cover 900; however, this not required. Many types of sealing materials can be used. Generally, the sealing material is a non-conductive material and is essentially inert to the components that are contact with the sealing material and/or are in close proximity to the sealing material. One non-limiting type of sealing material that can be used is a silicone-based material. The color of the sealing material when dry can be any color. In one non-limiting embodiment, the color of the dried sealing material is generally slightly opaque to clear so that if the sealing material inadvertently contacts one or more LEDs and/or light pipes, the sealing material will not fully impair the function of the LED and/or light pipe. The amount of sealing material inserted in the housing can be at least partially controlled so as to obtain the desired seal between the circuit board 770, compartment portion 710 and/or cover 900, and/or to inhibit or prevent the sealing material from flowing through and/or being forced through one or more gaps between the circuit board and cover 900, thereby inhibiting or prevent the sealing material from 1) contacting and/or covering one or more LEDs, light pipes, windows, and/or components on the top surface of the circuit board, and/or 2) contacting one or more under surfaces of cover 900 which could impair the opening of cover 900, if such opening was desired. As can be appreciated, the configuration of cavity 800 can be used to cause the sealing material to flow in one or more areas. As illustrated in FIG. 30, the location of opening 1130 is closer to the location of gaps 1110 and 1120; however, this is not required. In addition, the outer surface of battery cavity 714 can be used to create a small gap between the outer surface of battery cavity 714 and the bottom surface of circuit board 770. This small gap can be used to resist the flow of sealing material through the gap so that more flow of the sealing material is toward gaps 1110 and 1120 to achieve the desired sealing and/or to prevent too much sealing material from flowing into undesired locations; however, this is not required.

Referring again to FIG. 3, the one or more batteries 64 that are included in HUD assembly 24 are in power communication with one or more components in the display logic 62. As previously mentioned, batteries can be included in HUD assembly 24 or one or more batteries and/or power supplies can be located remotely from HUD assembly 24. In one non-limiting arrangement, the one or more batteries are in power communication with power monitoring circuit 80. The power monitoring circuit 80 and light sensor 78 are illustrated as being in communication with the controller 66. The controller 66 is illustrated as being in communication with transceiver 74, indicator(s) 76, and communications port 82. The controller 66 can also be in communication with the antenna 70; however, this is not required. As can be appreciated, the HUD assembly can include other or additional electronic components. Antenna 70 can be used to wirelessly send/receive data messages W between HUD assembly 24 and sensor controller assembly 28. As also can be appreciated, the connection arrangement for the one or more components can be the same or different from that illustrated in FIG. 3. As also can be appreciated, one or more electronic components can include two or more components.

The memory 68 in the HUD assembly can have the same or similar functions as memory 48 in controller 46 of the sensor controller assembly 28; however, this is not required. As such, memory 68 can be designed to store one or more software programs that are executed by the controller 66; however, this is not required. These one or more software programs can be used to control various aspects of operation of the display logic 62, sensor controller assembly 28, one or more other components of HUD assembly 24, and/or one or more other components of SCBA 10; however, this is not required. The memory 68 can also or alternatively be used to store various parameters of one or more components of the HUD assembly 24, sensor assembly 14, and/or one or more components of SCBA 10 (e.g., type, make, model of one or more components of the SCBA 10, service information of one or more components of the SCBA 10, length of use of one or more components of the SCBA 10, maintenance information of one or more components of the SCBA 10, presets and/or adjust parameters of one or more components of the SCBA 10, assembly and/or repair information of one or more components of the SCBA 10, etc.); however, this is not required. The memory 68 can also or alternatively be used to store various types of records regarding the function and/or operation of one or more components of the SCBA 10 (e.g., current information and/or historical information of battery status, rate of battery use, estimate remaining time of battery power, current information and/or historical information geographic location of SCBA 10 [e.g., GPS information, LAN information, etc], current information and/or historical information of pressure and/or sensor data, rate of use air consumption from breathing gas supply, estimate remaining air from breathing gas supply, current time, time correlation and/or geographic correlation to one or more types of data stored in memory 68, etc.); however, this is not required. When time information is stored in memory 68, the time information can be actual time and/or actual date information, and/or can be time relative to when one or more components of the SCBA have been activated and/or deactivated. Memory 68 can be a variety of different types of memory (e.g., RAM, ROM, flash memory, etc.). In one non-limiting arrangement, at least a portion of memory 68 includes flash memory and/or some other type of memory that can be reprogrammed using a programming device connected to one or more communication ports 82. The one or more communication ports 82 can be designed to accept one or more types of cables (e.g., tire wire, USB, serial cable, phone cable, ethernet cable, etc.). The programming device can be any number of devices such as, but not limited to, a portable computer (e.g., laptop computer, notebook computer, tablet computer, PDA device, Palm PC device, Blackberry device, etc.), desktop and/or mainframe computer, cell phone, etc.

Referring now to FIGS. 2 and 3, the transceiver assembly 32 can be in an off or low power mode condition until one or more signals are received from sensor message construction logic 30; however, this is not required. The transceiver assembly 32 can be powered by the one or more batteries 33 as previously discussed above. As can also be appreciated, transceiver assembly 32 can be in an off or low power mode until one or more signals are received from sensor message construction logic 30; however, this is not required. The sensor message construction logic 30 can be designed to send various data messages to HUD assembly 24. The sensor message construction logic 30 can be designed to send/receive data messages to/from the HUD assembly 24. These data messages between HUD assembly 24 and sensor message construction logic 30 can be conveyed by transceiver assembly 32. The transceiver assembly 32 can be designed to wirelessly transmit the appropriate data messages to the HUD assembly 24; however, it can be appreciated that one or more data messages can be sent by wire and/or cable. The one or more data messages sent from sensor controller assembly 28 to HUD assembly 24 can be used to supply information to HUD assembly to be processed by the HUD assembly, and/or be used to at least partially control one or more operations of the HUD assembly (e.g., control one or more indicators 76, etc.); however, this is not required. The transceiver assembly 32 can be designed to wait for a confirmation or acknowledgment from the HUD assembly 24 that one or more of the data messages were received by the HUD assembly; however, this is not required. In such an arrangement, the transceiver assembly 32 can be designed to wait for a confirmation or acknowledgment from the HUD assembly 24 before sending an acknowledgment to the sensor message construction logic 30, and/or sending additional data messages to HUD assembly 24; however, this is not required. The transceiver assembly 32 can be designed to resend one or more data messages, and/or run and/or activate one or more diagnostic sequences when confirmation or acknowledgment from the HUD assembly 24 has not been received after some period of time; however, this is not required. After a proper acknowledgment is received from the HUD assembly 24 within a predetermined time, the transceiver assembly 32 can be designed to send a proper acknowledgment to the sensor message construction logic 30; however, this is not required. Once the transceiver assembly 32 is linked to the HUD assembly 24, this link can be maintained for a short or long period of time. In one non-limiting arrangement, the link is maintained until a system shutdown or a loss of the wireless communication signal occurs. In another non-limiting arrangement, the system can remain in an active mode until the sensor message construction logic 30 removes the power enable signal to the transceiver assembly 32. The transceiver assembly 32 is generally designed to transmit the information to the HUD assembly 24 as needed and/or during certain time intervals. The sensor controller assembly 28 and HUD assembly 24 can be designed to repeat the wireless transmissions and acknowledgments as described above so long as a shutdown is not received; however, this is not required. After a shutdown is received, the process can be designed to restart with the transceiver assembly 32 initially disabled; however, this is not required.

Referring now to FIG. 3, power can be applied to the display logic 62 by use of one or more batteries; however, this is not required. For example, one or more batteries can be inserted into battery compartments 714 (See FIGS. 24 and 25). The display logic 62 can be designed to blink one or more of the indicator(s) 76 one or more times to indicate the batteries are installed; however, this is not required. As can be appreciated, some other indicator blink sequences, other type of visual indicators and/or audible indicators can be used to indicate that the batteries have been properly installed. Once power is provided to display logic 62, the display logic 62 can initially enter a carrier detect mode with current at a minimum level; however, this is not required. One or more indicator(s) 76 can be designed to remain off until a valid wired/wireless signal from the sensor controller assembly 28 is received; however, this is not required. For example, the transceiver 74 can be designed to look for a wireless signal at some select frequency; however, this is not required. The display logic 62 can be designed to enter a receive mode when a wireless signal is detected; however, this is not required. When the display logic 62 receives a valid command, the display logic 62 can be designed to send an acknowledgment of the received signal back to the sensor controller assembly 28; however, this is not required. The display logic 62 can be designed to remain linked to the sensor controller assembly 28 until shutdown or loss of signal; however, this is not required. In one non-limiting arrangement, display logic 62 can be designed to blink one or more indicator(s) 76 for some period of time and/or for some number of blinks, then blink one or more indicators individually one time at some frequency (e.g., 1-10 Hz, etc.) and twice at some duty cycle level (e.g., 50%, 75%, etc.) to indicate power up. As can be appreciated, some other indicator blink sequence, other type of visual indicator and/or audible indicator can be used to indicate power up of display logic 62. Once the display logic 62 is properly powered up, the display logic can then be designed to make a determination to enter a continuous or intermittent display mode based on instructions from the sensor controller assembly 28; however, this is not required. The display logic 62 can be designed to remain in one or more display modes so long as it is linked to the sensor controller assembly 28; however, this is not required. When the display logic 62, in intermittent display mode, receives the first signal related to breathing gas pressure from the sensor controller assembly 24, the display logic 62 can be designed to activate the appropriate pressure indicator(s) for some period of time (e.g., at least 10-30 seconds, etc.) and/or enter into some blink sequence; however, this is not required. When the display logic 62, in continuous display mode, receives the first signal related to breathing gas pressure from the sensor controller assembly 24, the display logic 62 can be designed to keep the appropriate pressure indicators illuminated at the proper intensity constantly, and/or constantly cause the one or more displays to blink; however, this is not required. The display logic 62 can be designed to automatically adjust the brightness of the indicator(s) based on a signal from the light sensor 78 indicating ambient light conditions; however, this is not required. As can be appreciated, the adjustment of the brightness of one or more indicator(s) can be manual; however, this is not required. As can also be appreciated, one or more functions of the HUD assembly and/or the sensor assembly can be at least partially activated, deactivate and/or controlled by one or more voice commands; however, this is not required.

When the HUD assembly is designed so that it can be in an intermittent display mode, and when the HUD assembly is in such a mode, the HUD assembly, after the minimum initial activation of the appropriate indicators, can be designed to have other appropriate indicators activated depending on certain conditions identified in the signal from the sensor controller assembly 28; however, this is not required. These conditions include, but are not limited to, some residual pressure in the breathing gas supply (e.g., above ¾ residual pressure, residual pressure between ¾ and ½, residual pressure between ½ and ¼, residual pressure between ¼ and a predetermined minimum pressure [e.g., 98 psi (0.7 MPa)], etc.), a low battery condition (e.g., 75% power, 50% power, 25%, power, 10% power, etc.), a loss of a wireless/wired link, a PASS pre-alert condition, a PASS alarm condition, and/or shutdown. For example, a signal that indicates a residual pressure in the breathing gas supply above ¾ when the display logic is in the intermittent display mode, the display logic 62 can be designed to repetitively illuminate green, yellow, amber, and/or red pressure indicators for some time period (e.g., 2-20 seconds, etc.) and then extinguish the indicators for some time period (e.g., 30-90 seconds) as long as the appropriate signal is received; however, this is not required. As can be appreciated, many other display sequences can be used when the display logic is in the intermittent display mode. In another and/or alternative example, a signal that indicates a residual pressure in the breathing gas supply between ¾ and ½ when the display logic is in the intermittent display mode, the display logic 62 can be designed to repetitively illuminate yellow, amber, and/or red indicators for some time period (e.g., 2-20 seconds, etc.) and then extinguish the indicators for some time period (e.g., 30-90 seconds) as long as the appropriate signal is received; however, this is not required. As can be appreciated, many other display sequences can be used when the display logic is in the intermittent display mode. In still another and/or alternative example, a signal that indicates a residual pressure in the breathing gas supply between ½ and ¼ when the display logic is in the intermittent display mode, the display logic 62 can be designed to repetitively illuminate amber and/or red indicators for some time period (e.g., 2-20 seconds, etc.) and then extinguish the indicators for some time period (e.g., 30-90 seconds) as long as the appropriate signal is received; however, this is not required. Alternatively, the display logic 62 can be designed to repetitively illuminate flash amber and red indicators at some frequency (e.g., 1-10 Hz, etc.) and at some duty cycle (e.g., 5-20%, etc.) for some period of time (e.g., 5-30 seconds, etc.) and then extinguish the displays for some time period (e.g., 20-90 second, etc.); however, this is not required. This sequence may be repeated as long as the appropriate signal is received; however, this is not required. As can be appreciated, many other display sequences can be used when the display logic is in the intermittent display mode. In yet another and/or alternative example, a signal that indicates a residual pressure in the breathing gas supply between ¼ and empty and/or some predetermined minimum pressure level when the display logic is in the intermittent display mode, the display logic 62 can be designed to repetitively illuminate the red indicator for some time period (e.g., 2-20 seconds, etc.) and then extinguish the indicators for some time period (e.g., 30-90 seconds) as long as the appropriate signal is received; however, this is not required. Alternatively, the display logic 62 can be designed to repetitively illuminate the red indicator at some frequency (e.g., 1-10 Hz, etc.) and at some duty cycle (e.g., 5-20%, etc.) for some period of time (e.g., 5-30 seconds, etc.) and then extinguish the displays for some time period (e.g., 20-90 second, etc.); however, this is not required. This sequence may be repeated as long as the appropriate signal is received; however, this is not required. As can be appreciated, many other display sequences can be used when the display logic is in the intermittent display mode. In still yet another and/or alternative example, a signal that indicates a residual pressure in the breathing gas supply is below some predetermined minimum pressure level when the display logic is in the intermittent display mode, the display logic 62 can be designed to repetitively illuminate or continuously illuminate the red indicator as long as the appropriate signal is received; however, this is not required. This sequence may be repeated as long as the appropriate signal is received; however, this is not required. As can be appreciated, many other display sequences can be used when the display logic is in the intermittent display mode.

The HUD assembly can also be designed to inform a user of low batter power; however, this is not required. In one example, a signal that indicates a low batter power (e.g., 1-3 hours of SCBA operation remain, etc.) when the display logic is in the intermittent display mode, can cause the display logic 62 to repetitively illuminate the low power indicator for some time period (e.g., 2-20 seconds, etc.) and then extinguish the indicators for some time period (e.g., 30-90 seconds) as long as the appropriate signal is received; however, this is not required. Alternatively, the display logic 62 can be designed to repetitively illuminate the low power indicator at some frequency (e.g., 1-10 Hz, etc.) and at some duty cycle (e.g., 5-20%, etc.) for some period of time (e.g., 5-30 seconds, etc.) and then extinguish the displays for some time period (e.g., 20-90 second, etc.); however, this is not required. This sequence may be repeated as long as the appropriate signal is received; however, this is not required. As can be appreciated, many other display sequences can be used when the display logic is in the intermittent display mode. The low battery indicator can be extinguished when the display logic 62 is in a sleep mode; however, this is not required. When the HUD assembly battery 64 becomes too low to provide reliable information when the display logic is in the intermittent display mode, the display logic 62 can be designed to flash the green, yellow, amber, and/or red pressure indicators at some frequency (e.g., 1-10 Hz, etc.) and at some duty cycle (e.g., 5-20%, etc.) for some period of time (e.g., 5-30 seconds, etc.) and then extinguish the displays for some time period (e.g., 20-90 second, etc.); however, this is not required. This sequence may be repeated as long as the appropriate signal is received; however, this is not required. This blinking indicator sequence can be different for the lower power signal than for the low air pressure signal so as to enable the use to distinguish these two different types of warnings; however, this is not required. As can be appreciated, many other display sequences can be used when the display logic is in the intermittent display mode. When the controller battery 64 power becomes too low to provide reliable information, a shutdown signal can be sent to sensor controller assembly 28 and/or occur in HUD assembly 24; however, this is not required.

When a loss of a link occurs while the display logic 62 is in an intermittent display mode, the display logic 62, after some period of time (e.g., 1-5 minutes, etc.), can be designed to repetitively scan the green yellow, amber, and red pressure indicators left to right or right to left at some frequency (e.g., 1-10 Hz) and extinguish them for some period of time (2-20 seconds, etc.); however, this is not required. If the link is not restored within some period of time (e.g., 2-10 minutes, etc.) a shutdown sequence can occur; however, this is not required. This blinking indicator sequence can be different from the blinking sequence for the lower power signal and/or low air pressure signal so as to enable the use to distinguish these different types of warnings; however, this is not required. As can be appreciated, many other display sequences can be used when the display logic is in the intermittent display mode.

When a PASS pre-alert condition occurs while the display logic 62 is in an intermittent display mode, the display logic 62 can be designed to repetitively flash the PASS indicator as some rate (e.g., 20-200 ms per second, etc.); however, this is not required. When a PASS alarm condition occurs while the display logic 62 is in an intermittent display mode, the display logic 62 can be designed to continuously illuminate the PASS indicator at full brightness, regardless of ambient light conditions; however, this is not required. As can be appreciated, many other display sequences can be used when the display logic is in the intermittent display mode.

Referring now to FIG. 4, one non-limiting embodiment for the configuration of antenna 52 and antenna 70 is an antenna that includes a loop stick antenna having multiple windings of wire wound around a ferrite core 84. As can be appreciated, one or more antenna can have a different configuration. As illustrated in FIG. 4, the ferrite core 84 is generally linear; however, it can be appreciated that other shapes can be used. The antenna in FIG. 4 includes wire windings 86, 88, and 90 that are wound directly on the ferrite core 84. Winding 88 can include a plurality of the windings, wherein a first winding can be wound directly on the ferrite core 84 and a second or subsequent winding can be wound over the first and/or previous windings. A similar arrangement can exist for windings 86 and/or 90. The type of wire for windings 86, 88 and/or 90 can be the same or different. The core material of core 84 and/or the cross-section size and/or shape of the core can be the same or vary along the longitudinal length of the core. Antenna 52 and/or antenna 70 can have a variety of inductance ratings (e.g., 95-140 mH, etc.) and a variety of resistances (e.g., 576+/−10% Ohms, etc.). The antenna illustrated in FIG. 4 can be used to provide a wireless link with characteristics of an inductive loop system; however, this is not required. The effective transmission range between the sensor controller assembly 28 and HUD assembly 24 generally falls off faster than for non-loop stick antennas. This characteristic can reduce cross-coupling between SCBAs 10. Paired antennas 52, 70 in mated sensor controller and HUD assemblies can provide wireless communication via magnetic inductive coupling; however, this is not required.

Referring now to FIG. 5, another exemplary embodiment of an antenna 52′ and/or antenna 70′ is illustrated. The antenna includes a loop stick antenna with multiple loops of wires wound around a nonlinear ferrite core 92. The nonlinear ferrite core 92 can be curved or arc-shaped as shown. In other non-limiting arrangement, other non-linear shaped ferrite cores can be used. In one non-limiting arrangement, windings 86, 88, and 90 can be wound directly on the non-linear ferrite core 84 in the same manner as described above for the linear ferrite core 84 of FIG. 4. The type of wire for windings 86, 88 and/or 90 in FIG. 5 can be the same or different. The core material of core 92 and/or the cross-section size and/or shape of the core can be the same or vary along the longitudinal length of the core. The inductance and/or resistance of the antenna of FIG. 5 can be the same or different from the inductance and/or resistance of the antenna of FIG. 4. The antenna configuration of FIG. 5 can be implemented in HUD assembly 24 and/or sensor controller assembly 28. Paired antennas 52′, 70′ in mated sensor controller and HUD assemblies can provide wireless communication via magnetic inductive coupling; however, this is not required.

Referring now to FIG. 6, another exemplary embodiment of an antenna 52″ and/or antenna 70″ is illustrated. The antenna includes a loop stick antenna with multiple loops of wires wound around multiple ferrite cores. Winding 86 can be wound around a first ferrite core 94, winding 88 can be wound around a second ferrite core 96, and winding 90 can be wound around a third ferrite core 98. The ferrite cores 94, 96, 98 can be generally linear; however, this is not required. For instance, any combination of the ferrite cores can be non-linear shaped. Winding 88 can include a plurality of the windings, wherein a first winding can be wound directly on the ferrite core 96 and a second or subsequent winding can be wound over the first and/or previous windings. A similar arrangement can exist for windings 86 and/or 90. The type of wire for windings 86, 88 and/or 90 can be the same or different. The core material of cores 94, 96, 98 and/or the cross-section size and/or shape of the cores can be the same or vary along the longitudinal length of the core. Antenna 52″ and/or antenna 70″ can have a variety of inductance ratings and a variety ofresistances. The antenna configuration of FIG. 6 can be implemented in HUD assembly 24 and/or sensor controller assembly 28. Paired antennas 52″, 70″ in mated sensor controller and HUD assemblies can provide wireless communication via magnetic inductive coupling; however, this is not required.

The individual windings 86, 88, 90 in the antennas illustrated in FIGS. 4-6 can be connected in various configurations and combinations to the antenna communication lines via a switching network; however, this is not required. The associated controller 46, 66 can be used to control the switching network and/or configure the antenna; however, this is not required. Thus, the actual antenna configuration can be programmable and/or can be tuned to optimize wireless communication via a maintenance procedure, and/or can be dynamically tuned to optimize wireless communication during operation; however, this is not required

The wireless communication system of the SCBA can include any antenna embodiment of FIGS. 4-6 in the sensor controller assembly and/or the HUD assembly in any combination. As can be appreciated, other antenna configurations can be used. In one non-limiting arrangement, the HUD assembly of the SCBA can include the antenna embodiment of FIG. 5 and the corresponding sensor controller assembly can include the antenna embodiment of FIG. 6; however, this is not required.

Referring now to FIG. 7, one non-limiting sensor data message sequence 100 that is transmitted from a sensor controller assembly of an SCBA to a HUD assembly is disclosed. The sensor data message sequence 100 includes an initialization portion 102, a preamble portion 104, a controller serial number portion 106, a data portion 108, a HUD serial number portion 110, and a block checksum (BCC) portion 112. The initialization portion 102 can include two 8-bit words; however, it can be appreciated that the initialization portion can include more or less bits and/or words. In this non-limiting arrangement, a valid initialization portion 102 can include 16 consecutive 0-level bits (i.e., 0000000000000000); however, it can be appreciated that one or more 1-level bits can be used. In another non-limiting arrangement, the preamble portion 104 can include one 8-bit word; however, it can be appreciated that the initialization portion can include more or less bits and/or more words. In this non-limiting arrangement, a valid preamble portion 104 can include an alternating sequence of 1-level and 0-level bits (i.e., 10101010); however, it can be appreciated that other 0-level and/or 1-level bit sequences can be used. In another non-limiting arrangement, the controller serial number portion 106 can include two 8-bit words; however, it can be appreciated that the initialization portion can include more or less bits and/or words. In this non-limiting arrangement, there are up to about 65,536 unique identifiers that can be used for the wireless controller assemblies. In another non-limiting arrangement, the data portion 108 can include two 8-bit words; however, it can be appreciated that the data portion can include more or less bits and/or words. In this non-limiting arrangement, embodiment, there are up to 65,536 unique combinations that can be used in the data portion 108. In another non-limiting arrangement, the HUD serial number portion 110 can include two 8-bit words; however, it can be appreciated that the HUD serial number portion can include more or less bits and/or words. In this non-limiting arrangement, there are up to about 65,536 unique identifiers for the wireless HUD assemblies. In another non-limiting arrangement, the BCC portion 112 can include one 8-bit word; however, it can be appreciated that the BCC portion can include more or less bits and/or more words. The value for the BCC portion 112 can be determined by performing an exclusive OR operation on the words making up the controller serial number portion 106, data portion 108, and HUD serial number portion 110; however, this not required. Generally, the one or two 8-bit words for the initialization portion 102, preamble portion 104, controller serial number portion 106, data portion 108, HUD serial number portion 110, and BCC portion 112 are different; however, this is not required. As can be appreciated, various logical function and/or a mathematical function can be used to determine the value for the BCC portion 112. As mentioned above, more or fewer bits can be used for any portion of the sensor data message sequence 100. In addition, it will be appreciated that one or more portions of sensor data message sequence 100 can be arranged in a different order, certain portions can be omitted, and/or additional portions can be added to the sequence. As can also be appreciated, the value of one or more portions can be cleared to define a particular situation; however, this is not required. For instance, the HUD serial number portion 110 can be cleared if the sensor controller assembly is transmitting the sensor data message sequence to any HUD assembly. As can be appreciated, there can be many other or additional examples of one or more portions being modified or cleared based on some a particular situation.

Referring now to FIG. 8, one non-limiting display data message sequence 120 transmitted from a HUD assembly of an SCBA to a sensor controller assembly is illustrated. The display data message sequence 120 includes an initialization portion 122, a preamble portion 124, a controller serial number portion 126, a data portion 128, a HUD serial number portion 130, and/or a block checksum (BCC) portion 132. In non-limiting arrangement, the initialization portion 122 can include one 8-bit word; however, it can be appreciated that the initialization portion 122 can include more or less bits and/or more words. In this non-limiting arrangement, a valid initialization portion 122 can include eight consecutive 0-level bits (i.e., 00000000); however, it can be appreciated that one or more 1-level bits can be used. In another non-limiting arrangement, the preamble portion 124 can include one 8-bit word; however, it can be appreciated that the preamble portion 124 can include more or less bits and/or more words. In this non-limiting arrangement, a valid preamble portion 124 can include an alternating sequence of 0-level bits and 1-level (i.e., 01010101); however, it can be appreciated that other 0-level and/or 1-level bit sequences can be used. In another non-limiting arrangement, the controller serial number portion 126 can include two 8-bit words; however, it can be appreciated that the controller serial number portion 126 can include more or less bits and/or words. In this non-limiting embodiment, there are up to about 65,536 unique identifiers for the wireless controller assemblies. In non-limiting arrangement, the data portion 128 can include one 8-bit word; however, it can be appreciated that the data portion 128 can include more or less bits and/or more words. In this embodiment, there are up to about 256 unique combinations that can be used in the data portion 128. In another non-limiting arrangement, the HUD serial number portion 130 can include two 8-bit words; however, it can be appreciated that the HUD serial number portion 130 can include more or less bits and/or words. In this non-limiting arrangement, there are up to about 65,536 unique identifiers for wireless HUD assemblies. In another non-limiting arrangement, the BCC portion 132 can include one 8-bit word; however, it can be appreciated that BCC portion 132 can include more or less bits and/or more words. The value for the BCC portion 132 can be determined by performing an exclusive OR operation on the words making up the controller serial number portion 126, data portion 128, and HUD serial number portion 130; however, this is not required. As can be appreciated, other logical functions and/or mathematical functions can be used to determine the value for the BCC portion 132. Generally, the one or two 8-bit words for the initialization portion 122, preamble portion 124, controller serial number portion 126, data portion 128, HUD serial number portion 130, and BCC portion 132 are different; however, this is not required. As mentioned above, more or fewer bits can be used for any portion of the display data message sequence 120. In addition, it will be appreciated that one or more portions of display data message sequence 120 can be arranged in a different order, certain portions can be omitted, and/or additional portions can be added to the sequence. As can also be appreciated, the value of one or more portions can be cleared to define a particular situation; however, this is not required. For instance, controller serial number portion 126 can be cleared if the HUD assembly is transmitting the display data message sequence to any sensor controller assembly. As can be appreciated, there can be many other or additional examples of one or more portions being modified or cleared based on some a particular situation.

With reference to flow charts illustrated in FIGS. 9-22, certain operations of the system are described with reference to corresponding flow charts. In the flow charts, the rectangular elements denote “processing blocks” and represent computer software instructions or groups of instructions. The diamond shaped elements denote “decision blocks” and represent computer software instructions or groups of instructions that affect the execution of the computer software instructions represented by the processing blocks. The remaining parallelogram shaped elements denoted “input or output blocks” and represent computer software instructions or groups of instructions that are either read data from various sources or send data to various sources. Alternatively, the processing, decision, and input and output blocks represent steps performed by functionally equivalent circuits such as a digital signal processor circuit or an application specific integrated circuit (ASIC). The flowchart does not depict syntax of any particular programming language. Rather, the flowchart illustrates the functional information one skilled in the art may use to fabricate circuits and/or to generate computer software to perform the processing of the system. It should be noted that many routine program elements, such as initialization of loops and variables and/or the use of temporary variables are not shown. It should also be appreciated that one or more steps in the flow charts are optional steps, some of which, but not all, are indicated below. It should be further appreciated that additional steps can be included in one or more of the flow charts below.

Referring now to FIG. 22, one non-limiting embodiment of a process 150 for displaying one or more conditions associated with an SCBA is illustrated. The wearer of the SCBA starts at 152 the display process. Next, a first data message based at least partially on one or more conditions associated with the breathing apparatus is received (154). At 156, the first data message is transmitted to a display assembly in a face mask of the breathing apparatus. Next, the display assembly receives the first data message and, in response, transmits a second data message to acknowledge receipt of the first data message (158). At 160, conditions associated with the breathing apparatus are displayed on the display assembly based at least in part on the first data message. Then, the process has reached its end (162). As can be appreciated, one or more of the above steps can include more than one data message.

Referring now to FIG. 9, one non-limiting embodiment of an initialization process 200 for a sensor controller assembly of an SCBA is illustrated. The initialization process 200 for the sensor controller assembly begins at 202. At 204, a battery can be inserted in one or more battery compartments of the sensor controller assembly. Next, at 212, the process can advance to a start sensor data message transmit process. One such non-limiting process is illustrated in FIG. 10.

Referring now to FIG. 10, there is illustrated one non-limiting embodiment of a sensor data message transmit process 230 in a sensor controller assembly of an SCBA. The sensor data message transmit process 230 begins at 232. At 236, a transmitter assembly or transceiver assembly in the sensor controller assembly can be used to receive a signal from a sensor message construction logic to enable power to the transceiver assembly (236); however, this is not required. Next, the process can determine if the sensor controller assembly is currently mated with a specific HUD assembly (240); however, this is not required. For example, the last HUD assembly with which the sensor controller assembly communicated can be considered its specific mate; however, this is not required. Moreover, a serial number of the specific mate can be stored in the transmitter assembly or transceiver assembly status flags and/or event log and/or some other storage location within the sensor controller assembly and/or at some other location in the SCBA. The transmitter assembly or transceiver assembly status flags and/or event log can also be programmed to identify a specific HUD assembly for the sensor controller assembly, for example, by serial number and/or some other identifier; however, this is not required. If a specific mate is identified, at 244, the transmitter assembly or transceiver assembly can be designed to receive a constructed sensor data message (e.g., see FIG. 8) from the sensor message construction logic; however, this is not required. As can be appreciated, a mate can be another SCBA, a command center, and/or some other unit. Next, the transmitter assembly or transceiver assembly acknowledgment and/or status message can be sent to the sensor message construction logic in response to successfully receiving the sensor data message (246). At 248, the transceiver assembly power can be set to high; however, this is not required. Next, the sensor data message can be wire/wirelessly transmitted from the transceiver assembly to the specific HUD assembly mated with the sensor controller assembly (250); however, this is not required. At 252, the process can be designed to wait for a predetermined time to determine if a display data message was received from the specific HUD assembly; however, this is not required. In one non-limiting arrangement, the predetermined time for the wait can be approximately one second; however, other time periods can be used. If a display data message is received, the process can be designed to advance to a routine to process the display data message (254, see FIG. 11).

At 240, if a specific mate is not identified, the process can advance to a low power search for mate process (256, see FIG. 14); however, this is not required. At 252, if a display data message is not received, the process can advance to 258 and can be designed to determine if a predetermined timer associated with multiple transmissions of a given sensor data message to a specific mate is expired. In one non-limiting arrangement, the predetermined timer can be approximately 12 seconds; however, it will be appreciated that other time periods can be used. If the predetermined timer is not expired, the process can be designed to return to 250 to re-transmit the sensor data message to the specific mate. Otherwise, if the predetermined timer is expired, the process can advance to a low power search for mate process (260, see FIG. 14).

Referring now to FIG. 11, one non-limiting arrangement for processing a display data message 310 in a sensor controller assembly of an SCBA is illustrated. The arrangement for processing a display data message 310 begins at 312. In one non-limiting arrangement, the display data message can include initialization, preamble, sensor controller serial no., data, HUD serial no., and/or checksum portions such as, but not limited to, those described above and shown in FIG. 8. In another non-limiting arrangement, the display data message can include the data portion with or without any combination of the above portions and/or other information portions. Before actually processing the display data message at 314, a transmitter assembly or transceiver assembly acknowledgment and/or status message can be sent by the transmitter assembly or transceiver assembly to the sensor message construction logic; however, this is not required. This transmission can be used to inform the sensor message construction logic that the display data message was received.

In the next step for processing a display data message 310, a checksum for the display data message can be calculated (316). In one non-limiting arrangement, the checksum can be calculated by performing an exclusive OR operation on words making up the sensor controller serial no., data, and/or HUD serial number portions and/or other information of the display data message. In another non-limiting arrangement, an additional or different logical function and/or a mathematical function can be used to calculate the checksum. At 318, the calculated checksum can be compared to the checksum portion of the display data message to determine if there is a checksum match. If the checksums match, the sensor controller assembly can construct a reply message (320). At 322, the reply message can be wired/wirelessly transmitted from the transmitter assembly or transceiver assembly to the specific HUD assembly mated with the sensor controller assembly. The reply message can provide an acknowledgment to the HUD assembly that the display data message was received; however, this is not required.

In the next step for processing a display data message 310, the process can begin analyzing the data portion of the display data message. At 324, the process can determine if the data portion includes an acknowledgment that a valid sensor data message was received by the HUD assembly. If an acknowledgment is included, transmitter assembly or transceiver assembly status flags and a transceiver assembly event log can be updated to reflect the acknowledgment (326). At 328, the process can determine if the data portion includes information indicating the HUD battery is low. If the HUD battery power is low, transmitter assembly or transceiver assembly status flags this event and a transceiver assembly event log can be updated to reflect the acknowledgment that the battery is low (338). This low battery information can be transmitted by a transmitter assembly or transceiver to the sensor message construction logic (330). This can be used to inform the sensor message construction logic of the content of the data portion of the display data message. In one non-limiting arrangement, the low battery information can be used to cause the HUD to enter into a low power mode, shut down, and/or to not display one or more data messages; however, this is not required. If the HUD battery is not low, a transmitter assembly or transceiver assembly acknowledgment and status message can be sent by the transmitter assembly or transceiver assembly to the sensor message construction logic (330). This can be used to inform the sensor message construction logic of the content of the data portion of the display data message. In another non-limiting arrangement, the process can be used to check for other information that can be provided in the data portion of the display data message; however, this is not required. At 332, the process can advance to a wireless communication system linked process (see FIG. 12). At 318, if the checksums do not match, the process can advance to a wireless communication system link lost process (334, see FIG. 13). Similarly, at 324, if the data portion of the display data message indicates that the HUD assembly could not acknowledge that a valid sensor data message was received, the process can advance to a wireless communication system link lost process (336, see FIG. 13).

Referring now to FIG. 12, one non-limiting arrangement for a wireless communication system linked process 350 in a sensor controller assembly of an SCBA is illustrated. The wireless communication system linked process 350 begins at 352. At 360, the transmitter assembly or transceiver assembly receives a constructed sensor data message from the sensor message construction logic. Next, a transmitter assembly or transceiver assembly acknowledgment and/or status message can be sent to the sensor message construction logic in response to successfully receiving the sensor data message (362); however, this is not required. At 364, the transceiver assembly power can be set to high; however, this is not required.

In the next step for the wireless communication system linked process 350, the sensor data message can be wired/wirelessly transmitted from the transmitter assembly or transceiver assembly to the specific HUD assembly mated with the sensor controller assembly (366). At 368, the process can wait for a predetermined time to determine if a display data message was received from the specific HUD assembly. In one non-limiting arrangement, the predetermined time to the wait can be approximately one second; however, other time periods can be used. If a display data message was received, the process can advance to a routine to process the display data message (370, see FIG. 11). At 368, if a display data message was not received, the process can advance to a wireless communication system link lost process (374, see FIG. 13).

Referring now to FIG. 13, one non-limiting arrangement of a wireless communication system link lost process 380 in a sensor controller assembly of an SCBA is illustrated. The wireless communication system link lost process 380 begins at 382. Next, the sensor data message can be wired/wirelessly transmitted from the transmitter assembly or transceiver assembly to the specific HUD assembly mated with the sensor controller assembly (384). At 386, the process can wait for a predetermined time to determine if a display data message was received from the specific HUD assembly. In one non-limiting arrangement, the predetermined time for the wait can be approximately one second; however, it can be appreciated that other time period can be used. If a display data message was received, the process can advance to a routine to process the display data message (388, see FIG. 11).

At 386, if a display data message was not received, the process can advance to 390 and to determine if a predetermined timer associated with multiple transmissions of a given sensor data message to a specific mate has expired. In one non-limiting arrangement, the predetermined timer can be approximately 60 seconds; however, it can be appreciated that other time period can be used. If the predetermined timer has not expired, the process can return to 384 to retransmit the sensor data message to the specific mate. Otherwise, if the predetermined timer has expired, the process can advance to a low power search for mate process (392, see FIG. 14).

Referring now to FIG. 14, one non-limiting arrangement for a low power search for mate process 400 in a sensor controller assembly of an SCBA is illustrated. The low power search for mate process 400 begins at 402. Typically, this process is used after a constructed sensor data message was already received by the transceiver assembly for transmission; however, this is not required. The process can be designed to determine if a constructed sensor data message was received (404); however, this is not required. If a constructed sensor data message was already received, any specific mate information in the sensor data message such as, but not limited to, the HUD serial number can be cleared from the message (406). In one non-limiting arrangement, the process of clearing the specific mate information can include setting the HUD serial number portion of the message to all zeroes or to some other value. In other non-limiting arrangement, such clearing can include setting any specific mate information in any other portion of the message to zero or deleting any specific mate information from any portion of the message. At 408, the transceiver assembly power can be set to low.

In the next step, low power search for mate process 400, the sensor data message can be wirelessly transmitted from the transmitter assembly or transceiver assembly to any HUD assembly that can be mated with the sensor controller assembly (410). At 412, the process can be designed to wait for a predetermined time to determine if a display data message was received from any. HUD assembly. In one non-limiting arrangement, the predetermined time for the wait can be approximately one second; however, it can be appreciated that other time periods can be used. If a display data message is received, the process can be designed to advance to a routine to process the display data message (414, see FIG. 11).

At 404, if a constructed sensor data message was not already received, the transceiver assembly can be designed to receive a constructed sensor data message from the sensor message construction logic with the specific mate information (i.e., HUD serial number portion) cleared (418). Next, a transceiver assembly acknowledgment and/or status message can be sent to the sensor message construction logic in response to successfully receiving the sensor data message (420) and the process can advance to 408 and continue as described above. At 412, if a display data message was not received, the process can advance to 422 and can determine if a predetermined timer associated with multiple transmissions of a given sensor data message to any mate at low power has expired. In one non-limiting arrangement, the predetermined timer can be approximately 12 seconds; however, it can be appreciated that other time periods can be used. If the predetermined timer is not expired, the process can be designed to return to 410 to re-transmit the sensor data message to any mate. Otherwise, if the predetermined timer is expired, the process can be designed to advance to a high power search for mate process (424, see FIG. 15). As can be appreciated, a high powered search for a mate can always be conducted.

Referring now to FIG. 15, one non-limiting arrangement for a high power search for mate process 430 in a sensor controller assembly of an SCBA is illustrated. The high power search for mate process 430 begins at 432. At 434, the transceiver assembly power can be set to high. Next, the sensor data message can be wirelessly transmitted from the transmitter assembly or transceiver assembly to any HUD assembly that can be mated with the sensor controller assembly (436). At 438, the process can be designed to wait for a predetermined time to determine if a display data message is received from any HUD assembly. In one non-limiting arrangement, the predetermined time for the wait can be approximately one second; however, it can be appreciated that other time periods can be used. If a display data message is received, the process can be designed to advance to a routine to process the display data message (440, see FIG. 11).

At 438, if a display data message is not received, the process can be designed to advance to 442 and can be designed to determine if a predetermined timer associated with one or more multiple transmissions of a given sensor data message to any mate at high power has expired. In non-limiting arrangement, the predetermined timer can be approximately 12 seconds; however, it can be appreciated that other time periods can be used. If the predetermined timer is not expired, the process can be designed to return to 436 to re-transmit the sensor data message to any mate; however, this is not required. Otherwise, if the predetermined timer is expired, at 444, a transmitter assembly or transceiver assembly non-acknowledgment and/or status message can be sent to the sensor message construction logic in response to not being able to find a mating HUD assembly. At 446, power to the transceiver assembly can be disabled; however, this is not required. Next, the process can be designed to advance to the start sensor data message transmit process (448, see FIG. 10) can immediately or after some time period repeat the message transmit process; however, this is not required. Information regarding failure to connect to a mate and/or information regarding connection to a mate can be displayed to the user of the SCBA; however, this is not required.

Referring now to FIG. 16, one non-limiting arrangement for an initialization process 2000 for a HUD assembly of an SCBA is illustrated. The initialization process 2000 begins at 2002. At 2004, a battery can be inserted in one or more battery compartments of the HUD assembly. Thereafter, the HUD assembly can be designed to flash all of its indicators on and off five times to show that power was just applied (2006). This can also serve as a visual check to determine if the indicators are operating properly. As can be appreciated, other visual sequences can be used to function as a visual check to determine if the indicators are operating properly. At 2008, HUD status flags and a HUD event log can be updated to, for example, reflect that power was just applied to the HUD assembly; however, this is not required.

The next step of the initialization process 2000 can be used to determine if the battery or power source in the HUD assembly is low (i.e., less than or equal to a predetermined limit) (2010). In one non-limiting arrangement, the predetermined low power limit for the HUD battery can be a level at which there are two operating hours remaining; however, it can be appreciated that a longer or shorter operating range can be used to define the predetermined low power limit. If the HUD battery is greater than the predetermined low power limit, at 2012 the process can be designed to advance to a start sensor data message receive process (see FIG. 17). At 2010, if the HUD battery is low, the process can be designed to determine if the HUD battery is so low (i.e., less than or equal to a second predetermined limit) that the HUD assembly needs to shutdown (2014). In one non-limiting arrangement, the second predetermined low power limit for the sensor controller battery can be, for example, 2.2 volts for a 3 volt power source; however, it can be appreciated that the second predetermined low power limit can be a different value based on a different power source voltage rating and/or based on a different ratio to the power source voltage rating. The second predetermined lower limit is generally less than the first predetermined limit. If the HUD battery is low, but greater than the second predetermined low power limit, at 2016 the process can be designed to update the HUD status flags and/or event log for a HUD battery low condition; however, this is not required. Next, the process can be designed to light a battery low indicator in the HUD assembly (2018) and advance to the start sensor data message receive process (2012, see FIG. 17); however, this is not required. However, it can be appreciated that if a low battery condition is detected, the process will not advance to step 2012 and can be designed to enter a shutdown mode after some predetermined period of time; however, this is not required. At 2014, if the sensor controller battery is less than or equal to the second predetermined low power limit, the process can transition to a shutdown mode (2020). The operation of the HUD assembly stops at 2022. Operation of the HUD assembly can be restarted, for example, when at least one battery is replaced.

With reference to FIG. 17, one non-limiting arrangement of a sensor data message receive process 2030 in a HUD assembly of an SCBA is illustrated. The sensor data message receive process 2030 begins at 2032. Next, the process can be designed to determine if a sensor data message was received from a sensor controller assembly (2036). If a sensor data message was received, the process can be designed to transition to a receive mode (2038). Next, the process can be designed to call a construct display data message process (2040, see FIG. 18) which can be designed to return a display data message (e.g., see FIG. 8); however, this is not required. At 2042, the display data message can be wired/wirelessly transmitted to the specific sensor controller assembly mated with the HUD assembly (i.e., the sensor controller assembly from which the sensor data message was received). Next, the process can be designed to call up a routine to process the sensor data message that was received (2044, see FIG. 19). At 2046, the process can be designed to wait a predetermined time to determine if a sensor controller reply message was received from the specific sensor controller assembly; however, this is not required. In one non-limiting arrangement, the predetermined time for the wait can be approximately one second; however, it can be appreciated that other time periods can be used. If a sensor controller reply message was received, the process can be designed to advance to a wireless communication system linked process (2048, see FIG. 17); however, this is not required.

At 2036, if a sensor data message was not received, the process can be designed to advance to 2050 and can be designed to determine if a predetermined receive timer has expired; however, this is not required. In one non-limiting arrangement, the predetermined receive timer can be approximately six seconds; however, it can be appreciated that other time periods can be used. If the predetermined receive timer is expired, the process can be designed to transition to asleep mode (2052) to conserve power; however, this is not required. Next, the process can be designed to determine if a predetermined sleep timer is expired (2054); however, this is not required. In one non-limiting arrangement, the predetermined sleep timer can be approximately ten seconds; however, it can be appreciated that other time periods can be used. If the predetermined sleep timer is expired, the process can be designed to return to 2036 and continue as described above; however, this is not required. Otherwise, the process can be designed to continue in the sleep mode until the predetermined sleep timer expires.

At 2046, if a sensor controller reply message was not received, the process can be designed to advance to 2056 and can be designed to determine if a predetermined transmit timer associated with multiple transmissions of a given display data message to a specific mate has expired; however, this is not required. In non-limiting arrangement, the predetermined transmit timer can be approximately 12 seconds; however, it can be appreciated that other time periods can be used. If the predetermined transmit timer has not expired, the process can be designed to return to 2042 to re-transmit the display data message to the specific mate; however, this is not required. Otherwise, the process can be designed to return to 2036 and continue as described above. At 2050, if the predetermined receive timer is not expired, the process can be designed to return to 2036 and continue as described above.

Referring now to FIG. 18, one non-limiting arrangement of a construct display data message process 2060 in a HUD assembly of an SCBA is illustrated. The construct display data message process 2060 begins at 2062. In one non-limiting arrangement, the display data message can include initialization, preamble, sensor controller serial no., data, HUD serial no., and/or checksum portions such as those described above and shown in FIG. 8. In another non-limiting arrangement, the display data message can include the data portion with or without any combination of the above portions and/or other information portions. At 2064, a checksum for the sensor data message that was received can be calculated. In non-limiting arrangement, the checksum can be calculated by performing an exclusive OR operation on words making up the sensor controller serial no., data, and/or HUD serial number portions of the sensor data message. In another non-limiting arrangement, a different or additional logical function and/or a mathematical function can be used to calculate the checksum. At 2066, the calculated checksum can be compared to the checksum portion of the sensor data message to determine if there is a checksum match. If the checksums match, the process can be designed to update the HUD status flags and/or event log for acknowledgment that a valid sensor data message was received (2068); however, this is not required.

Next, the process can be designed to determine if the battery or power source in the HUD assembly is low (i.e., less than or equal to a first predetermined low power limit) (2070); however, this is not required. In one non-limiting arrangement, the first predetermined low power limit for the HUD battery can be a level at which there are two operating hours remaining; however, it can be appreciated that a longer or shorter operating range can be used to define the predetermined low power limit. If the HUD battery is greater than the first predetermined low power limit, at 2072, the data portion of the display data message can be constructed based on the HUD status flags. At 2074, the process can be designed to identify a serial number of the specific sensor controller assembly mated with the HUD assembly and/or a serial number of the HUD assembly; however, this is not required. For example, these serial numbers can be stored in the HUD status flags and/or event log and/or in some other storage location within the HUD assembly.

Next, a checksum portion for the display data message can be calculated (2076). In one non-limiting arrangement, the checksum portion can be calculated by performing an exclusive OR operation on words making up the sensor controller serial no., data, and/or HUD serial number portions of the display data message. In another non-limiting arrangement, an additional or different logical function and/or mathematical function can be used to calculate the checksum portion. At 2078, the display data message can be built from the data portion and/or other information. In one non-limiting arrangement, the sensor data message can include initialization, preamble, sensor controller serial no., data, HUD serial no., and/or checksum portions. In another non-limiting arrangement, the display data message can include the data portion with or without any combination of these portions and/or other information portions. Next, the construct display data message process can be designed to return to the process from which it was called (2080); however, this is not required.

At 2066, if the checksums do not match, the process can be designed to update the HUD status flags and/or event log for non-acknowledprient, indicating that the sensor data message received was not valid (2082); however, this is not required. Next, the process can be designed to advance to 2072 and continue as described above; however, this is not required.

At 2070, if the HUD battery is low, the process can be designed to determine if the HUD battery is on low (i.e., less than or equal to a second predetermined low power limit) that the HUD assembly needs to shutdown (2084); however, this is not required. In one non-limiting arrangement, the second predetermined low power limit for the sensor controller battery can be, for example, 2.2 volts for a 3 volt power source; however, it can be appreciated that the second predetermined low power limit can be a different value based on a different power source voltage rating and/or based on a different ratio to the power source voltage rating. The second predetermined low lower limit is generally less than the first predetermined limit. If the HUD battery is low, but greater than the second predetermined limit, at 2086, the process can be designed to update the HUD status flags and/or event log for a HUD battery low condition; however, this is not required. Next, the process can be designed to light a battery low indicator in the HUD assembly (2088); however, this is not required. Next, the process can be designed to advance to 2072 and continue as described above. However, it can be appreciated that if a low battery condition is detected, the process will not advance to step 2072 and can be designed to enter a shutdown mode after some predetermined period of time. At 2084, if the sensor controller battery is less than or equal to the second predetermined low power limit, the process can transition to a shutdown mode (2090); however, this is not required. The operation of the HUD assembly stops at 2092. Operation of the HUD assembly can be restarted, for example, when at least one battery is replaced.

Referring now to FIG. 19, one non-limiting arrangement for processing a sensor data message 2100 in a HUD assembly of an SCBA is illustrated. The processing of a sensor data message 2100 begins at 2102. In one non-limiting arrangement, the sensor data message can include initialization, preamble, sensor controller serial no., data, HUD serial no., and/or checksum portions such as those described above and shown in FIG. 7. In another non-limiting arrangement, the sensor data message can include the data portion with or without any combination of the above portions and/or other information portions.

The next step for processing a sensor data message 2100 can include the analyzing of the data portion of the sensor data message. At 2104, the process can be designed to determine if the data portion includes a shutdown command. If the data portion does not include a shutdown command, at 2106, the process can be designed to identify an indicator control command based on the data portion of the sensor data message; however, this is not required. Next, the HUD status flags and/or event log can be updated based on the indicator control command (2108). At 2110, the HUD indicators can be lighted/extinguished based on the HUD status flags and/or event log. Next, the process sensor data message routine can be designed to return to the process from which it was called (2112). At 2104, if the data portion includes a shutdown command, the process can be designed to advance to the start of the sensor data message receive process (2116, see FIG. 17) to repeat the message receive process; however, this is not required.

Referring now to FIG. 20, one non-limiting arrangement for wireless communication system linked process 2120 in a HUD assembly of an SCBA is illustrated. The wireless communication system linked process 2120 begins at 2122. Next, the process can be designed to determine if a sensor data message was received from a sensor controller assembly (2126); however, this is not required. If a sensor data message was received, the process can be designed to call a construct display data message process (2128, see FIG. 18) which can be designed to return a display data message (e.g., see FIG. 8); however, this is not required. At 2130, the display data message can be wired/wirelessly transmitted to the specific sensor controller assembly mated with the HUD assembly (i.e., the sensor controller assembly from which the sensor data message was received); however, this is not required. Next, the process can be designed to call a routine to process the sensor data message that was received (2132, see FIG. 19); however, this is not required. At 2134, the process can be designed to wait for a predetermined time to determine if a sensor controller reply message was received from the specific sensor controller assembly; however, this is not required. In one non-limiting arrangement, the predetermined time for the wait can be approximately one second; however, it can be appreciated that other time periods can be used. If a sensor controller reply message was received, the process can be designed to return to 2126 and continue as described above.

At 2126, if a sensor data message was not received, the process can be designed to advance to 2136 and can be designed to determine if a predetermined receive timer associated with receiving messages from a specific mate is expired; however, this is not required. In one non-limiting arrangement, the predetermined receive timer, may be approximately five minutes; however, it can be appreciated that other time periods can be used. If the predetermined receive timer is expired, the wireless communication system link between the HUD assembly and the specific sensor controller assembly may be lost (2138). Next, the process can be designed to advance to the start sensor data message receive process (2140, see FIG. 17) to repeat the message receive process; however, this is not required.

At 2134, if a sensor controller reply message was not received, the process can be designed to advance to 2142 and can be designed to determine if a predetermined transmit timer associated with multiple transmissions of a given display data message to a specific mate is expired; however, this is not required. In one non-limiting arrangement, the predetermined transmit timer may be approximately 12 seconds; however, it can be appreciated that other time periods can be used. If the predetermined transmit timer is not expired, the process can be designed to return to 2130 to re-transmit the display data message to the specific mate; however, this is not required. At 2142, if the predetermined receive timer is expired, the wireless communication system link between the HUD assembly and the specific sensor controller assembly may be lost (2144); however, this is not required. Next, the process can be designed to advance to the start sensor data message receive process (2146, see FIG. 17) to repeat the message receive process; however, this is not required.

While the present invention has been illustrated by the description of embodiments thereof, and while the embodiments have been described in considerable detail, it is not the intention of the applicants to restrict or in any way limit the scope of the appended claims to such detail. Additional advantages and modifications will readily appear to those skilled in the art. For example, the data can be from any of several sensors including biometric, temperature, gas detection, or others; the display can be any of several visual indicators including but not limited to LEDs, liquid crystal displays (LCDs), incandescent lamps, or others; the information can also be conveyed as an audible or spoken message through prerecorded or speech synthesis means. Therefore, the invention, in its broader aspects, is not limited to the specific details, the representative apparatus, and illustrative examples shown and described. Accordingly, departures may be made from such details without departing from the spirit or scope of the general concept.

While various aspects of the invention are described and illustrated herein as embodied in combination in the various arrangements of the invention, these various arrangements can be realized in many alternative embodiments, either individually or in various combinations and sub-combinations thereof. Unless expressly excluded herein all such combinations and sub-combinations are intended to be within the scope of the present invention. Still further, while various alternative embodiments as to the various aspects and features of the invention, such as alternative materials, structures, configurations, methods, devices, software, hardware, control logic and so on may be described herein, such descriptions are not intended to be a complete or exhaustive list of available alternative embodiments, whether presently known or later developed. Those skilled in the art may readily adopt one or more of the aspects, concepts or features of the invention into additional embodiments within the scope of the present invention even if such embodiments are not expressly disclosed herein. Additionally, even though some features, concepts or aspects of the invention may be described herein as being a preferred arrangement or method, such description is not intended to suggest that such feature is required or necessary unless expressly so stated. Still further, exemplary or representative values and ranges may be included to assist in understanding the present invention however, such values and ranges are not to be construed in a limiting sense and are intended to be critical values or ranges only if so expressly stated.

1-93. (canceled)

94. A method of sealing an electronic enclosure comprising:
a) providing a housing that includes at least one printed circuit board and at least one gap between the printed circuit board and an inner wall of said housing;
b) providing a sealing material;
c) applying said sealing material to said at least one printed circuit board; and,
d) causing a portion of said sealing material to flow toward and at least partially into said at least one gap to form a seal between said at least one printed circuit board and said inner wall of said housing, said sealing material caused to flow toward and to at least partially flow into said at least gap by applying pressure to said sealing material as said sealing material is applied to said at least one printed circuit board to thereby force a portion of said sealing material to flow toward and at least partially into said at least one gap.

95. The method as defined in claim 94, including the step of orienting said housing in a manner to enable gravity to force a portion of said sealing material to flow toward and at least partially into said at least one gap.

96. The method as defined in claim 94, wherein said printed circuit board includes a top and bottom side, said step of applying said sealing material on said bottom side of said printed circuit board, said step of inhibiting said sealing material from flowing onto more than about 50% of said top surface of said printed circuit board.

97. The method as defined in claim 94, including the step of inhibiting said sealing material from flowing onto no more than about 30% of said top surface of said printed circuit board.

98. The method as defined in claim 97, wherein said sealing material flows onto no more than about 5% of said top surface of said printed circuit board.

99. The method as defined in claim 94, wherein a cavity exists between said printed circuit board of said inner wall of said housing, said step of including the applying of said sealing material into said cavity until at least about 10% of said cavity is filled with said sealing material.

100. The method as defined in claim 94, wherein over 50% of said cavity is filled with said sealing material.

101. The method as defined in claim 94, wherein said step of applying includes at least partially injecting said sealing material into at least one opening in said housing.

102. The method as defined in claim 94, wherein said sealing material includes a silicone-based material.

103. A method of displaying at least one condition associated with a breathing apparatus to a user of the breathing apparatus comprising:
a) wirelessly transmitting at least one data message to a display assembly in a face mask of the breathing apparatus;
b) receiving at least one data message on said display assembly;
c) acknowledging receipt of said at least one data message by said display assembly wirelessly transmitting at least one other data message;
d) displaying at least one condition associated with said breathing apparatus based at least partially on said at least one data message received by the display assembly.

104. The method as defined in claim 103, including the step of obtaining, receiving, and combinations thereof at least one data message from at least one component on said breathing apparatus, said at least one data message based at least partially on at least one condition associated with the breathing apparatus.

105. The method as defined in claim 103, wherein said step of acknowledging receipt of said at least one data message includes transmitting said at least one other data message to at least one other component of said breathing apparatus.

106. The method as defined in claim 103, wherein said step of transmitting includes obtaining information by a sensor assembly and transmitting at least one data message from said sensor assembly to said display assembly.

107. The method as defined in claim 103, including the step of verifying a connection between said display assembly and at least one other component of said breathing apparatus, said step of verifying including informing a user of the breathing apparatus that a connection has been established, a connection is currently being maintained, a connection has been lost, a connection cannot be made, and combinations thereof.

108. The method as defined in claim 108, wherein said step of verifying a connection between said display assembly and at least one other component of said breathing apparatus includes attempting to establish or reestablish said connection when said connection has been lost, said connection cannot be made, and combinations thereof.

109. The method as defined in claim 108, wherein said step of verifying a connection between said display assembly and at least one other component of said breathing apparatus includes entering into a lower power mode at some period of time, after some number of attempts to form a connection, or combinations thereof when said connection cannot be made, said connection cannot be reestablished, and combinations thereof.

110. The method as defined in claim 103, including the step of conducting a low power search, a high power search, or combinations thereof for a mate to said breathing apparatus.

111. The method as defined in claim 103, including the step of causing a low power mode to be entered by at least one component of said breathing apparatus, causing a power shutdown of at least one component of said breathing apparatus, and combinations thereof when said at least one data message indicates a low power supply.

112. The method as defined in claim 106, wherein said display assembly is at least partially connected to said mask to enable a user to at least partially view said display when wearing said mask, said sensor assembly designed to receive information based at least partially on at least one condition obtained by at least one sensor and to at least partially wirelessly transmit a first data message to said display assembly, said display assembly designed to at least partially wirelessly transmit a second data message to said sensor assembly to acknowledge receipt of said first data message.

113. The method as defined in claim 103, wherein said display assembly includes at least one display arrangement and a display housing, said at least one display arrangement designed to at least partially display information to a user about at least one condition of said breathing apparatus, said at least one display arrangement at least partially secured to said display housing, said display housing including first and second end portions and a connecting portion connected therebetween, said first end portion including a first battery compartment portion, said second end portion including a second battery compartment portion, each of said battery compartment portions designed to contain at least one battery.

114. The method as defined in claim 113, wherein at least one display arrangement is located on said first battery compartment portion and at least one display arrangement is located on said second battery compartment portion, said at least one display arrangement on said first battery compartment portion including at least one display designed to at least partially indicate a remaining air supply of said breathing apparatus, said at least one display arrangement on said second battery compartment portion including at least one display designed to at least partially indicate a different type of information from said at least one display on said first battery compartment portion.

115. The method as defined in claim 103, wherein said display assembly comprises at least one display arrangement and a display housing, said at least one display arrangement designed to at least partially display information to a user about at least one condition of said breathing apparatus, said at least one display arrangement at least partially secured to said display housing, said at least one of said display arrangement including at least one display and a viewing arrangement, said at least one display arrangement designed to at least partially display info, illation to a user about at least one condition of said breathing apparatus, said view arrangement designed to enable a nonuser of said breathing apparatus to view information about at least one condition of said breathing apparatus when a user is wearing said mask.

116. The method as defined in claim 115, wherein said viewing arrangement enables the nonuser view information about at least one condition of said breathing apparatus through said shield of said mask.

117. The method as defined in claim 106, wherein said display assembly and said sensor assembly each include at least one antenna, each of said antennas having a configuration to provide wireless communication between said display assembly and said display assembly via magnetic inductive coupling, at least one of said antennas include a core and a plurality of wire windings about at least a portion of said core, at least two of said windings spaced from one another on said core, said core having a non-linear shape along a longitudinal axis of said core.

118. The method as defined in claim 103, wherein said display system is designed to be at least partially connected to an interior surface of the mask by a retaining member, said retaining member designed to at least partially engage a region at least closely adjacent to a rim around a periphery of said protective shield inside the mask to at least partially secure said display housing to the mask, said retaining member includes first and second leg portions and a center portion connected therebetween, said first and second leg portions designed to at least partially conform to a shape of said protective shield around at least a portion of the periphery of said protective shield.
